SEQUENCE LISTING (15 sequences)

SEQ ID NO: 1 - DNA, 12 bp, Artificial Sequence
  Description of Artificial Sequence Synthetic oligonucleotide
tcggacattcaa

SEQ ID NO: 2 - DNA, 12 bp, Artificial Sequence
  Description of Artificial Sequence Synthetic oligonucleotide
tcagacattcaa

SEQ ID NO: 3 - DNA, 6 bp, Artificial Sequence
  Description of Artificial Sequence Synthetic oligonucleotide
ctgagt

SEQ ID NO: 4 - DNA, 6 bp, Artificial Sequence
  Description of Artificial Sequence Synthetic oligonucleotide
ccggag

SEQ ID NO: 5 - DNA, 32 bp, Artificial Sequence
  Description of Artificial Sequence Synthetic oligonucleotide
ttcaaacaacccccttaaaactatctcaaacg

SEQ ID NO: 6 - DNA, 32 bp, Artificial Sequence
  Description of Artificial Sequence Synthetic oligonucleotide
tctgagtaacccccccggagctatcctgagtg

SEQ ID NO: 7 - DNA, 16 bp, Artificial Sequence
  Description of Artificial Sequence Synthetic oligonucleotide
tcctggcatatcatca

SEQ ID NO: 8 - DNA, 10236 bp, Artificial Sequence
  Description of Artificial Sequence Synthetic polynucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtcgatcgactacgtcgttaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttccgtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccgcggctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgacaagcttgcatgcgctagcgtcgatcgactctagaggatcagaaaattatcgccataaaagacagaataagtcatcagcggttgtttcatttcctatattttttttttatttttttattttttaataagggaaaatttaacgtctaaggatacagaagattgttagcacattaaagtaataaaggcttaagtagtaagtgccttagcatgttattgtatttcaaaggacataatctaaaataataacaatatcatttctcacaagttattcaattttcttttttttttctaataatatcaagaatgtattatttgtttgacataagtcaactaatttatttaatatgctggattaatcttgcagacatgtaaattaacaagttttagtcaaataacgttgaagtttcaatgaactcaaataatttctctttttttttatataaccatatgtctaatctgatttatattttccgcagggatcaactgaagttatgacatttggattggatcacttataaccttggtcgccaaataatacaaaaatcagcgttataaaacaaagaaggtttttgttaagaaattaatcctctttcttgataagaaagttgaaccgaaattgcagatactgatatatgaaaataatacccacaattttgggaatagcgcaagcctcaatttaaacaataggtgaggacacatgataatgacctcaatgattgttagaagaaaagagcctcattacaaaatcgaaaaatgaatggttgggtacaagtttccaaaacatggtaaagtggactttgcgtatgagacgtaaatagaaaaaaacacttgttatatgttttctagaattattgttgtctctttatggttggatgatgcaaaatagtaatttcggttagttgctgtaaaacaccacgagacaaatagatatggatatttattaaatcaggaaaaacgtaactctcggctactggatggttcagtcacccaacgattactggggagagaaaacagggcaaaagcaaagcttaaaggaatccgattgtcattcggcaatgtgcagcgaaactaaaaaccggataatggacctgttaatcgaaacattgaagatatataaaggaagaggaatcctggcatatcatcaggaaactttttaaattttattctatttttctcttgggcaatttttgctttgctgtttggtttgaggtcagtcgccaaaggcaatagcattgtgtgcatgtgtgcatggttaaaatgaaaagttggctaacaacatttttattcaaaaatcattaacatacaaatctaatcaaaaacaaaatgaatatagtaagcaacaagaattaaatcatttttttttcattgttttaattttaatgagtaattcctttcttcaattccttattttttcatccttccattcattcattcatgcattccatcattcctccttgatgcaatggatcgcatggatatacatacattgatttgtgaatcgtgtgcatctattgatggatgggagttgtatgtgtttgtgtgatgagggtttgttgaagcaaggcattgtcgaggatgccctctattttgtgttgtgttttagattaaaagtttaatgtgtttatctctttaatatacattgttacacaaaatctctgtttatgatattcatttcgttcctatagtttattcgaatgaattgaattgaattgactagactgactatgcataccaatcttgttgcttctattgtttaacgaaatacgcatagaatgctcggattgtcgactttatcatagttttacgaagtgaaatttttttaaatgctgcactcacatactgactgtgtgatgtgaaagatgaaagtgtcatatcaaatttaatcaaatgaacccaagggagagtgaatattatgaattcactcatccttgatgttggcagaacgagtcgttttgtctgcatatctgagtaacccccccggagctatcctgagtgaaactaaactcgggcatgcatttcaacgttttgcatggtctaatcattttttgcattgactatatctttatttgcatttcactatattgttttgcactttactagatcgtttttgcatctatggtacgttttgcactaatgaattttttgcactcactcaatttaaactgcttccaagaataaaacgtttttatttttatttttttttcttttcctttgccgtccgttgtttgtcaccctcaactacacaaaatatcgactccgtgactgtcatgttttatctttcacacgaatgacgatgaaatgtgacagtttatctggtcaggatgattgtacagcctgggccttggtttgcatacaattgaacgaattgtgagtgatttaatggtggttaatcttagaaatactataatctacttcaaccaattctattacttcattttttgctttgctgatacggcatggttgcctcatgtgtaggacatttgtctgacaaaccaattatccctacacgggaagagaattccttgggttaaacggcatgtggtcattatcaaggtttcgttaacttttgtcccacaggatgaggttgccaaatcgtaataccagcctgggtctcaagagagaaaatgcgacaagggatgcatcggcaaatgccaactcaaagatgtcacttttacagtattcatgagcaatgtggtggtgatagactcaagtaactgtgttaaaattaagacaggggcctgctaaaatgaacttacgaataaatcgctttttccttcggacattcaaatcagtaagtcaaagtgtgaaagcgtcattggttgtgttattcgagacttgtaagtcggcagtaaatctatctgtagcgagtgtgtttgagaaaggaggaaatttttgcattgttcattagcagacttgacatttaccgtgtaggtatttaaggtggcaggcttgccagctagaaatttcagtgtgaaaaaaaaaataaaagtcttcaaaactggtcgtaaagcggccgcctgcagctcgagcccgggacgtatatctctttttaaacttaaacctgaaggtggctttatccttgagtctataattgttttagattccactcttctatcatttactttgcaacgttctttatttttaatagattgattttttaaaccctataattccggttgtttataaaatgtaagagctatttgacttaaagcgaaaaattgcataatataagcagaggatagtttatgtaggagataatgtaatctgctctataaaactgtcaaattgtctgtcagaagcacaacattaataagcagtccgctttcttactttaaattcgattaaacaaatatatgcgacgagtagaaataaaagcaaacttcaacctttggttcaacatatgctttctgccgttgcgtgtacaaacctatacgtgcacaacgttgtttaaggtgtttctttgacaatactctcaccatataaagattacgatttttttagaagagatgactaattacgcgatacctaaaaaatttcactctaagcgaatctaagatgtcctaagtttcgcatatgtattttggcgaaaattgatgccaaatcaatattaaacaaagaaaaacagtgaatagtacagcaaacttgaaaaatgaaaatggtacattgcgccatttttcgagttactagtgagctcgaattcgagtctaactccttaaccactcggacatagtgacttatctgacactcattgtaaattaatatatatataggcattttgtttagttaaaggtacttaagtaattagtataaacgaaccaattttataatcaggaagttaagtgaatggtagcacatgtcgtaaaaattgtgaatttttattgaataatattttaaatacaagcctttctagactaggtatactcataaacatatatgagcaaaaggatagaggagattacattgcatcttctacaaatttatttattgccctttactgaaaaattaaataatgagtactaaatgataaaaagcgctcagtacaagaaagattgcaaaaatattgcattcttcatgaattaaagttgcatataaggcatattgaaagtaatagtactaaaacagcagttagcgaaaattaatagaattatattcgcaagacaattgtgacattaaattaaaaaattgtagaatttttactatcctctttaacgccatgagcctttataaaaaggttaaattagttttaacattctttttttgagtaagagtttacaatttatcaaaacctgtgttattatattcattagtttcaatttattagcatctagagaaaaatcaattggcagttacattgttggaatttatgaagaaaaagaatctacaacggagaataagttgctgatcgctttccctaaaattgtatattttgctgagcttattttgacatttcgttgaagttttctctaattcgcattcattttaagtaaaacaatgagaaataaaattacaaaaaatacaaattaaaatacaatttttagctataattatagacgatgcccttgtatcccattctgtctcgcttgcccctactttttatcttttatataccataatgaacgctgccgctactaaccataccccgattttacatttcggactcccaaggacgtacaaaatagaaaactatagaaaaaaataatcagaaaatagcatgtcatctctttgtaaaacgcgtttgcaagaagaaaggaaacaatggagaagagatcatccatttgtatgtaaattttagtaaacttgaagaaatcactaacaacttctcttacttagggattctatgcaaaaccttgtaaatcatctgatggaggactcgatttaatgaattggaaggttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 9 - DNA, 26 bp, Artificial Sequence
  Description of Artificial Sequence Synthetic oligonucleotide
ggaagaggaatcctggcatatcatca

SEQ ID NO: 10 - DNA, 1643 bp, Artificial Sequence
  Description of Artificial Sequence Synthetic polynucleotide
ggaaactttttaaattttattctatttttctcttgggcaatttttgctttgctgtttggtttgaggtcagtcgccaaaggcaatagcattgtgtgcatgtgtgcatggttaaaatgaaaagttggctaacaacatttttattcaaaaatcattaacatacaaatctaatcaaaaacaaaatgaatatagtaagcaacaagaattaaatcatttttttttcattgttttaattttaatgagtaattcctttcttcaattccttattttttcatccttccattcattcattcatgcattccatcattcctccttgatgcaatggatcgcatggatatacatacattgatttgtgaatcgtgtgcatctattgatggatgggagttgtatgtgtttgtgtgatgagggtttgttgaagcaaggcattgtcgaggatgccctctattttgtgttgtgttttagattaaaagtttaatgtgtttatctctttaatatacattgttacacaaaatctctgtttatgatattcatttcgttcctatagtttattcgaatgaattgaattgaattgactagactgactatgcataccaatcttgttgcttctattgtttaacgaaatacgcatagaatgctcggattgtcgactttatcatagttttacgaagtgaaatttttttaaatgctgcactcacatactgactgtgtgatgtgaaagatgaaagtgtcatatcaaatttaatcaaatgaacccaagggagagtgaatattatgaattcactcatccttgatgttggcagaacgagtcgttttgtctgcatatctgagtaacccccccggagctatcctgagtgaaactaaactcgggcatgcatttcaacgttttgcatggtctaatcattttttgcattgactatatctttatttgcatttcactatattgttttgcactttactagatcgtttttgcatctatggtacgttttgcactaatgaattttttgcactcactcaatttaaactgcttccaagaataaaacgtttttatttttatttttttttcttttcctttgccgtccgttgtttgtcaccctcaactacacaaaatatcgactccgtgactgtcatgttttatctttcacacgaatgacgatgaaatgtgacagtttatctggtcaggatgattgtacagcctgggccttggtttgcatacaattgaacgaattgtgagtgatttaatggtggttaatcttagaaatactataatctacttcaaccaattctattacttcattttttgctttgctgatacggcatggttgcctcatgtgtaggacatttgtctgacaaaccaattatccctacacgggaagagaattccttgggttaaacggcatgtggtcattatcaaggtttcgttaacttttgtcccacaggatgaggttgccaaatcgtaataccagcctgggtctcaagagagaaaatgcgacaagggatgcatcggcaaatgccaactcaaagatgtcacttttacagtattcatgagcaatgtggtggtgatagactcaagtaactgtgttaaaattaagacaggggcctgctaaaatgaacttacgaataaatcgctttttccttcggacattcaa

SEQ ID NO: 11 - DNA, 192 bp, Artificial Sequence
  Description of Artificial Sequence Synthetic polynucleotide
tcggacattcaaatcagtaagtcaaagtgtgaaagcgtcattggttgtgttattcgagacttgtaagtcggcagtaaatctatctgtagcgagtgtgtttgagaaaggaggaaatttttgcattgttcattagcagacttgacatttaccgtgtaggtatttaaggtggcaggcttgccagctagaaatttc

SEQ ID NO: 12 - DNA, 24 bp, Artificial Sequence
  Description of Artificial Sequence Synthetic primer
gatttcttcttccatggtcttgtc

SEQ ID NO: 13 - DNA, 19 bp, Artificial Sequence
  Description of Artificial Sequence Synthetic primer
gttggcacaaacgacggcc

SEQ ID NO: 14 - DNA, 31 bp, Artificial Sequence
  Description of Artificial Sequence Synthetic oligonucleotide
tcctggcatatcatcaggaaactttttaaat

SEQ ID NO: 15 - DNA, 80 bp, Artificial Sequence
  Description of Artificial Sequence Synthetic oligonucleotide
aatttcagtgtgaaaaaaaaaataaaagtcttcaaaactggtcgtaaagcggccgcctgcagctcgagcccgggacgtat

(19) United States
(12) Patent Application Publication
(10) Pub. No.: US 2023/0265425 A1
(43) Pub. Date: Aug. 24, 2023

(54) lncRNA-REGULATED GENE EXPRESSION SYSTEM

(71) Applicant: MEMORIAL SLOAN KETTERING CANCER CENTER, New York, NY (US)

(72) Inventor: Angad GARG, New York, NY (US)

(21) Appl. No.: 18/004,028

(22) PCT Filed: Jul. 1, 2021
(86) PCT No.: PCT/US2021/040051

§ 371 Date: Dec. 30, 2022

Related U.S. Application Data
(60) Provisional application No. 63/047,628, filed on Jul. 2, 2020.

(51) Int. Cl. C12N 15/113; C12N 15/81

(52) U.S. Cl. CPC C12N 15/113; C12N 15/81; C12N 2320/50

(57) ABSTRACT

The present disclosure provides gene expression systems and methods for producing and/or overexpressing heterologous polypeptides in eukaryotic cells. Kits for practicing the methods are also disclosed.

### CROSS-REFERENCE TO RELATED APPLICATIONS

This application is a U.S. National Stage Application of PCT/US2021/040051, filed Jul. 1, 2021, which claims the benefit of and priority to U.S. Provisional Patent Application No. 63/047,628, filed Jul. 2, 2020, the contents of which are incorporated by reference herein in its entirety.

### STATEMENT OF GOVERNMENT SUPPORT

This invention was made with government support under GM052470 and GM126945 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 31, 2021, is named 115872-0918 SL.txt and is 19,467 bytes in size.

### TECHNICAL FIELD

The present technology relates generally to gene expression systems and methods for producing and/or overexpressing heterologous polypeptides in eukaryotic cells. Kits for practicing the methods are also disclosed.

### BACKGROUND

The following description of the background of the present technology is provided simply as an aid in understanding the present technology and is not admitted to describe or constitute prior art to the present technology.

The fission yeast Schizosaccharomyces pombe is an excellent model organism for the study of eukaryotic cellular physiology. Regulatable gene expression systems are a major tool in understanding the function of individual genes and their products. Rapid induction kinetics, reproducible and titratable induction levels, and low reagent costs are advantageous features of expression systems for use in basic research and for industrial-scale protein production. Limitations of currently available overexpression systems in fission yeast include: delay in expression after induction; narrow dynamic range; self-limiting gene expression; and system-wide changes due to induction conditions.

Thus, there is an urgent need for gene expression systems that possess rapid induction kinetics, broad dynamic range, and tunable expression.

### SUMMARY OF THE PRESENT TECHNOLOGY

In one aspect, the present disclosure provides an expression system comprising a nucleic acid sequence, wherein the nucleic acid sequence includes (a) a nc-tgp1 long noncoding RNA (lncRNA) gene that is operably linked to a thiamine responsive expression control sequence, and (b) a gene of interest that is operably linked to a tgp1 gene promoter, wherein the thiamine responsive expression control sequence is located upstream of the tgp1 gene promoter. In some embodiments, the gene of interest may be endogenous or heterologous. Additionally or alternatively, in some embodiments, the gene of interest encodes a protein, an enzyme, a structural polypeptide, a toxin, a fusion protein, an antibody agent, a drug, a cytokine, an enzyme inhibitor, a growth factor, a signaling protein, a catalytic RNA, or an inhibitory RNA (e.g., miRNA, siRNA, sgRNA, antisense oligonucleotide). In certain embodiments, the gene of interest is pho1, fkh2 or sak1.

Examples of suitable thiamine responsive expression control sequences include, but are not limited to a nmt1 promoter (Basi G, Schmid E, Maundrell K. (1993) Gene 123:131-136; Maundrell K. (1990) J Biol Chem 265:10857-10864), a Y. lipolytica P3 promoter (Walker et al., Appl Environ Microbiol. 2020 Feb; 86(3): e02299-19), and a Pichia pastoris THI11 promoter (Landes et al., Biotechnol Bioeng (2016) 113(12):2633-2643). In certain embodiments, the nmt1 promoter comprises the nucleic acid sequence TCCTGGCATATCATCA (SEQ ID NO: 7) or GGAAGAGGAATCCTGGCATATCATCA (SEQ ID NO: 9).

Additionally or alternatively, in some embodiments, the nc-tgp1 lncRNA gene comprises the nucleic acid sequence of SEQ ID NO: 10.

In any of the preceding embodiments of the expression system, the nc-tgp1 lncRNA gene comprises a cluster of DSR elements. In certain embodiments, the cluster of DSR elements comprises the nucleic acid sequence TTCAAACAACCCCCTTAAAACTATCTCAAACG (SEQ ID NO: 5). In other embodiments, the cluster of DSR elements is mutated and comprises the nucleic acid sequence CTGAGT (SEQ ID NO: 3) and/or CCGGAG (SEQ ID NO: 4). Additionally or alternatively, in some embodiments, the cluster of DSR elements comprises the nucleic acid sequence TCTGAGTAACCCCCCCGGAGCTATCCTGAGTG (SEQ ID NO: 6).

Additionally or alternatively, in some embodiments, the tgp1 gene promoter comprises the nucleic acid sequence of SEQ ID NO: 11. In certain embodiments of the expression system disclosed herein, the tgp1 gene promoter comprises a Pho7 DNA binding domain. The Pho7 DNA binding domain may comprise the nucleic acid sequence of TCGGACATTCAA (SEQ ID NO: 1) or TCAGACATTCAA (SEQ ID NO: 2).

The expression system of the present technology can be introduced as a linear construct, or a circular vector, which can be incorporated and inherited as a transgene integrated into the host genome, or as an extrachromosomal expression vector. The expression vector may be a DNA or RNA vector. Additionally or alternatively, in some embodiments, the expression vector is a plasmid, a cosmid, a bacmid, a bacterial artificial chromosome (BAC), a yeast artificial chromosome (YAC), or a viral vector.

In another aspect, the present disclosure provides a host cell comprising any and all embodiments of the expression system disclosed herein. The expression systems of the present technology may be used to induce/repress expression of a gene of interest in eukaryotic cells.

Additionally or alternatively, in some embodiments, the host cell is a Saccharomyces yeast, a Schizosaccharomyces yeast, a Candida yeast, a Pichia yeast, a Hansenula yeast, a Trichosporon yeast, a Brettanomyces yeast, a Pachysolen yeast, a Yamadazyma yeast, a Kluyveromyces yeast, or a Yarrowia yeast. Non-limiting examples of yeast cells include, but are not limited to, Saccharomyces cerevisiae, Schizosaccharomyces pombe, Candida shehatae, Pichia stipites, Kluyveromyces marxianus, Kluyveromyces lactis and the like.

In one aspect, the present disclosure provides a method for overexpressing a heterologous polypeptide in a eukaryotic cell comprising contacting any and all embodiments of the host cell disclosed herein with an effective amount of thiamine, wherein the heterologous polypeptide is encoded by the heterologous gene of the expression system of the present technology. In another aspect, the present disclosure provides a method for decreasing expression of a heterologous polypeptide in a eukaryotic cell comprising culturing any and all embodiments of the host cell in a thiamine-free medium, wherein the heterologous polypeptide is encoded by the heterologous gene of the expression system of the present technology. Additionally or alternatively, in some embodiments, the methods of the present technology further comprise detecting activity and/or expression levels of the heterologous polypeptide.

In any and all embodiments of the methods disclosed herein, the activity and/or expression levels of the heterologous polypeptide are detected via a functional catalytic assay (e.g., acid phosphatase activity assay), a phenotypic assay, a colorimetric assay, enzyme-linked immunosorbent assays (ELISA), dot blotting, immunofluorescence, fluorescent microscopy, immunoprecipitation, immunoelectrophoresis, flow cytometry, western blotting, HPLC, or mass-spectrometry.

Also provided herein are kits comprising any and all embodiments of the expression system disclosed herein, and instructions for use.

### BRIEF DESCRIPTION OF THE DRAWINGS

FIG. 1A shows: (top) a schematic illustration of the lncRNA-regulated thiamine-inducible system using Pho1 acid phosphatase as a reporter (pTIN-pho1), where transcription start sites (TSS) are shown as bent arrows, the positions of the nc-tgp1 TSS, nc-tgp1 lncRNA DSR mutations, tgp1 promoter Pho7 DNA binding site and tgp1 TSS are indicated, the distance between the nc-tgp1 TSS and the −1 nucleotide relative to the tgp1 translation start site is indicated by the bracket, and the pho1 ORF is denoted by a horizontal arrow in the direction of mRNA synthesis; and (below) the mechanism of the lncRNA-regulated thiamine-inducible system, where the thiamine-starved condition (−Thiamine) is the repressed state, thiamine-replete condition (+Thiamine) is the induced state for pho1 expression, and the two predominant nc-tgp1 lncRNA isoforms are indicated.

FIG. 1B shows the relative acid phosphatase activities of pho4Δ pho1Δ cells transformed with promoter-less pTIN-pho1 or pTIN.pho1. Single colonies of pho4Δ pho1Δ cells transformed with promoter-less pTIN pho1 or pTIN pho1 were pooled (≥20) and grown in Leu⁻ PMG medium. Cells were diluted in Leu⁻ PMG medium without or with 15 μM thiamine, and acid phosphatase activity was measured after incubation at 30° C. for 21 hours. The acid phosphatase activity is the average±SEM from three independent cultures.

FIG. 1C shows the photographs of serially diluted cultures of pho4Δ pho1Δ cells bearing the pTIN empty vector or pTIN pho1 in agar medium containing phosphatase probe α-naphthyl phosphate. A representative culture of pho4Δ pho1Δ cells bearing the pTIN empty vector or pTIN-pho1 were grown in Leu⁻ PMG medium without thiamine at 30° C. The cultures were adjusted to A600 of 0.1 and five-fold serial dilutions were spotted on Leu⁻ PMG agar medium without or with 15 μM thiamine and incubated at 30° C. for 3 days. The cells were overlaid with 1% agarose containing 0.015% a-naphthyl phosphate and 0.15% Fast Blue B Salt in 0.1 M sodium acetate (pH 4.2). The plates were photographed after incubation for 2 min at room temperature.

FIG. 2A shows the kinetics of induction by the lncRNA-regulated thiamine-inducible system, demonstrated by the change of phosphatase activity of pho4Δ pho1Δ cells bearing pTIN-pho1 plasmids over time. Single colonies of pho4Δ pho1Δ cells transformed with the pTIN pho1 plasmid were pooled (>20) and grown at 30° C. in Leu⁻ PMG without thiamine. Acid phosphatase activity was measured from an aliquot of the culture (time-0) and the remaining culture was adjusted to 15 μM thiamine. Acid phosphatase activity was measured at the indicated times after the addition of thiamine. Each datum is the average ±SEM from three independent cultures.

FIG. 2B shows the kinetics of induction by the lncRNA-regulated thiamine-inducible system, demonstrated by the change of pho1 mRNA levels of pho4Δ pho1Δ cells bearing pTIN-pho1 plasmid over time. pho4Δ pho1Δ A cells bearing the pTIN-pho1 plasmid was grown as described in FIG. 2A and aliquots were harvested before or at the indicated times after the addition of thiamine (time-0). Total RNA prepared from harvested samples was analyzed by reverse transcription primer extension using a mixture of radiolabeled primers complementary to the pho1 (top panel) or the act1 (bottom panel) mRNAs. The reaction products were resolved by denaturing urea-PAGE and visualized by autoradiography. The images shown in the top and bottom panels are from a single exposure of one gel. For conciseness of the figure, the intervening lanes of the gel were cropped (represented by the thin line separating the 3 and 10-hour samples). The positions and sizes (in nucleotides) of DNA markers are indicated on the left. Fold-induction reflects the ratio of the act1-normalizedpho1 signal at the indicated time to time-0.

FIG. 3A shows the effects of thiamine concentration on Pho1 expression as indicated by Pho1 activity. pho4Δ pho1Δ cells bearing the pTIN-pho1 plasmid were propagated at 30° C. in Leu⁻ PMG medium lacking thiamine. The cells were then diluted and grown for 20-23 hours in Leu⁻ PMG medium with the indicated thiamine concentration. The acid phosphatase activity is the average ±SEM from three independent cultures. The data were fit to a dose-response model (_{EC50} shift) in Graphpad Prism with a goodness of fit correlation coefficient of 0.99, Hill Slope of 2.29, and dose of half maximal response (EC50) of 0.069±0.005 μM thiamine.

FIG. 3B shows a comparison of the lncRNA-regulated thiamine-inducible system to the thiamine-repressible nmt1 expression system. Single colonies of pho4Δ pho1Δ cells transformed with the indicated plasmids were pooled (≥20), where the pTIN-pho1 or the pREP(3×; 41×; 81×)-pho1 bearing cells were grown in Leu− PMG medium lacking thiamine or containing 15 μM thiamine, respectively. Pho1 expression was induced by adjusting thiamine concentration to 15 μM for the pTIN-pho1 bearing cells or by pelleting cells, washing twice with water and resuspending in Leu− PMG medium lacking thiamine for the pREP-series-pho1 bearing cells. Acid phosphatase activity was measured in repressed conditions (time-0) and at the indicated times after induction. Each data point is the average±SEM from three independent cultures. The data were fit to a sigmoidal model (Boltzmann) in Graphpad Prism with a goodness of fit correlation coefficient of 0.99 for each induction curve.

FIG. 4A shows the schematic illustration of the lncRNA-regulated thiamine-inducible regulatory region with a mutated Pho7 binding site fused to the pho1 ORF. The base specific change is underlined. Figure discloses SEQ ID NOS 1-2, respectively, in order of appearance.

FIG. 4B shows the relative acid phosphatase activities of pho4Δ pho1Δ cells transformed with pTIN7m or pTIN7m-pho1. Single colonies of pho4Δ pho1Δ cells transformed with pTIN7m or pTIN7m-pho1 were pooled (>20) and grown at 30° C. in Leu⁻ PMG medium. The cultures were diluted and grown for 22 hours in Leu⁻ PMG medium with the indicated thiamine concentration and acid phosphatase activity was measured. Each datum is the average±SEM of three independent cultures.

FIG. 5A shows cell growth inhibition in the presence of thiamine when the lncRNA-regulated tgp1 promoter was on (Right), but not in the absence of thiamine when the lncRNA-regulated tgp1 promoter was off (Left).

FIG. 5B shows septation index (percentage of cell bearing a single septum) of cells bearing either the pTIN or pTIN-sak1 plasmid at different times after thiamine induction. WT cells bearing either the pTIN empty vector or pTIN-sak1 plasmid were grown at 30° C. in Leu⁻ PMG medium lacking thiamine. An aliquot of the cultures was fixed (time 0) and the remaining volume of cultures was adjusted to 15 μM thiamine. The cultures were incubated at 30° C. and an aliquot of each culture was fixed at the indicated times. Fixed samples were processed for DAPI and Calcofluor White staining and the proportion of septated cells was quantified by fluorescence microscopy. The total number of cells counted for each sample is indicated above the bar datum. The Z-test two tailed p value comparing the proportion of total septated cells of pTIN and pTIN-sak1 samples are—0 hour: 0.53, 4 hour: 1.86×10⁻², 8 hour: 1.18×10⁻⁵, and 24 hour: 9.18×10⁻²⁴.

FIG. 5C shows exemplary fluorescence images (DAPI and Calcofluor) of cells bearing the indicated plasmids (described in FIG. 5B) under de-repressed (+thiamine) conditions at 24 h. Arrows indicate examples of cells with aberrant mitosis.

FIG. 6 shows the pTIN/pTIN7m plasmid map. Features of the pTIN/pTIN7m vector are depicted. The positions of the indicated restriction sites are shown. The sequences of the following regions are shown at the bottom-left—the nmt1 promoter fused to the nc-tgp1 lncRNA transcription start site (TSS) and, the tgp1 5′ UTR with the multiple cloning site polylinker (MCS), where the black bent arrow marks the initiating nucleotide of the lncRNA or the mRNA as indicated. The base specific changes for mutations in the DSR element cluster are indicated at the bottom. The DNA sequences of the WT or mutant (mut) Pho7 DNA binding site present in the pTIN or pTIN7m vector, respectively, are indicated at the bottom-right. Figure discloses SEQ ID NOS 14-15, 5, 1-4 and 3, respectively, in order of appearance.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995)PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Disclosed herein are gene expression systems, wherein a thiamine responsive promoter (e.g., nmt1 promoter) regulates the synthesis of a lncRNA nc-tgp1 nucleic acid that is located directly upstream of a gene of interest that is operably linked to a tgp1 promoter. The addition of thiamine quickly halts synthesis of the nc-tgp1 lncRNA, thereby de-repressing the gene of interest.

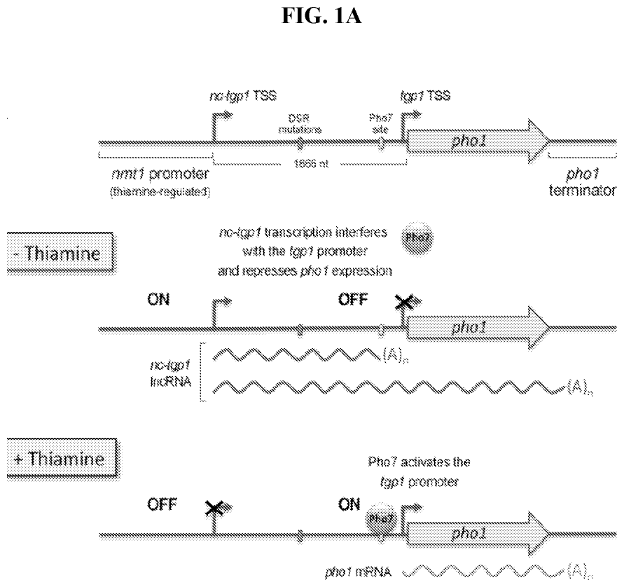

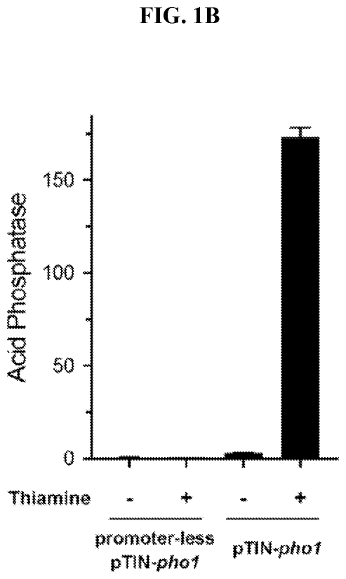

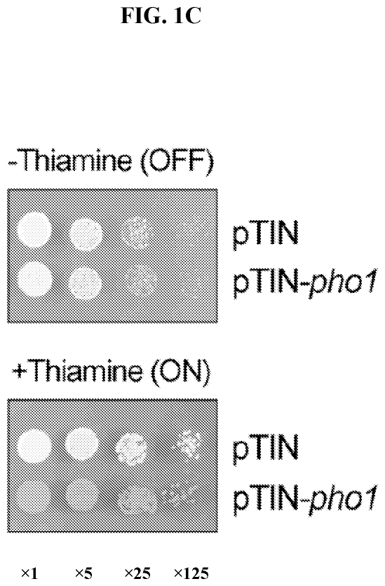

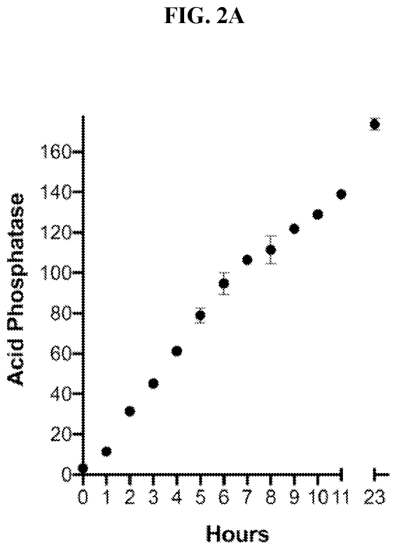

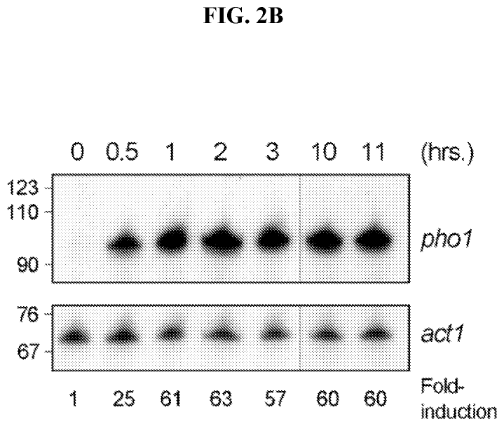

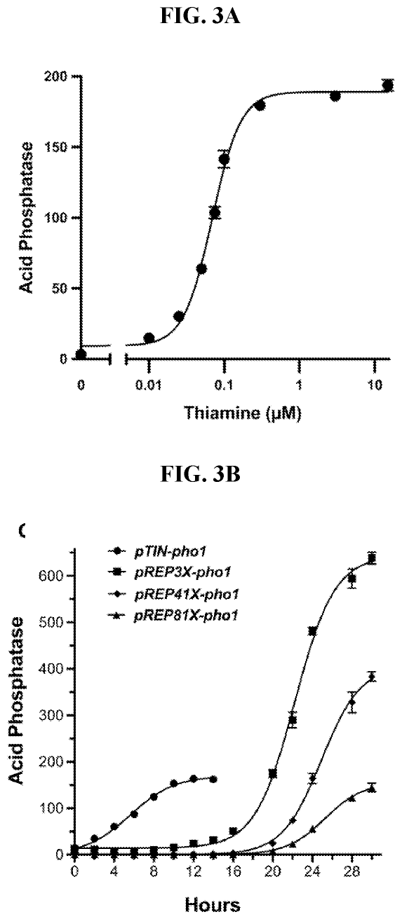

One advantage of the gene expression system of the present technology is the rapid kinetics of lncRNA-regulated gene expressions system. Target mRNA levels can reach its peak by 1 hour after induction (see FIG. 2B), which makes it possible to follow the effects of overproducing a gene product within one generation time of the host cell life cycle. The target protein is translated continuously and accumulates progressively after the induced steady-state mRNA level is achieved. (FIGS. 2A, 3B). The pTIN system also has a broad dynamic range, with as much as a 60-fold transcriptional induction observed when by the addition of thiamine. See FIGS. 1B, 2B. The level of protein expression can be tuned by varying the thiamine concentration (FIG. 3A). The gene expression system has a clear advantage of induction kinetics over the nmtl-based expression system as evidenced by the induction of half-maximal target protein levels 16.6 h, 19.3 h, or 19.7 h earlier than the pREP3X, pREP41X, or pREP81X expression vectors, respectively (FIG. 3B). Induction by simple addition of thiamine to the medium is less cumbersome than the standard nmt1 promoter-driven overexpression system, which necessitates harvesting, washing, and resuspending cells in thiamine-free medium. The pTIN system is modular, that is, the nmt1 promoter guides the expression of a repressive lncRNA that in turn controls the expression of the target gene. In principle, any one component may be changed to influence the expression level of the system; for example, the pTIN7m vector has a Pho7 binding site mutation in the tgp1 promoter, the effect of which is to reduce basal expression in the absence of thiamine and dampen the extent of protein overproduction in the presence of thiamine. This variant has potential applications in cases where tighter repression is called for, for example, when working with endogenous or heterologous proteins that are toxic at high gene dosage (i.e., when expressed from multicopy fission yeast plasmids).

Accordingly, the regulatable gene expression system is modular, tunable to fine-tune the target gene expression levels, and having a fast and broad dynamic range. The regulatable gene expression system of the present technology are useful in methods for regulated expression of target genes in eukaryote cells. Accordingly, the various aspects of the present technology relate to the preparation, characterization, and manipulation of a regulatable expression vector and a host cell comprising the same.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As generally understood, a “codon” is a series of three nucleotides (triplets) that encodes a specific amino acid residue in a polypeptide chain or for the termination of translation (stop codons). There are 64 different codons (61 codons encoding for amino acids plus 3 stop codons) but only 20 different translated amino acids. The overabundance in the number of codons allows many amino acids to be encoded by more than one codon. Different organisms (and organelles) often show particular preferences or biases for one of the several codons that encode the same amino acid. The relative frequency of codon usage thus varies depending on the organism and organelle. In some instances, when expressing a exogenous gene in a host organism, it is desirable to modify the gene sequence so as to adapt to the codons used and codon usage frequency in the host. In particular, for reliable expression of heterologous genes it may be preferred to use codons that correlate with the host's tRNA level, especially the tRNA's that remain charged during starvation. In addition, codons having rare cognate tRNA's may affect protein folding and translation rate, and thus, may also be used. Genes designed in accordance with codon usage bias and relative tRNA abundance of the host are often referred to as being “optimized” for codon usage, which has been shown to increase expression level. Optimal codons also help to achieve faster translation rates and high accuracy. In general, codon optimization involves silent mutations that do not result in a change to the amino acid sequence of a protein.

As used herein, a “control” is an alternative sample used in an experiment for comparison purpose. A control can be “positive” or “negative.”

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “effective amount” refers to a quantity sufficient to achieve a desired effect, e.g., an amount which results in the overexpression of a heterologous protein.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, the term “gene” means a segment of DNA that contains all the information for the regulated biosynthesis of an RNA product, including promoters, exons, introns, and other untranslated regions that control expression. When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA. A “native gene” or “endogenous gene” refers to a gene that is native to the host cell with its own regulatory sequences whereas an “exogenous gene” or “heterologous gene” refers to any gene that is not a native gene, comprising regulatory and/or coding sequences that are not native to the host cell. In some embodiments, an exogenous gene may comprise mutated sequences or part of regulatory and/or coding sequences. In some embodiments, the regulatory sequences may be heterologous or homologous to a gene of interest. A heterologous regulatory sequence does not function in nature to regulate the same gene(s) it is regulating in the transformed host cell. “Coding sequence” refers to a DNA sequence coding for a specific amino acid sequence or RNA sequences. As used herein, “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, ribosome binding sites, translation leader sequences, RNA processing site, effector (e.g., activator, repressor) binding sites, stem-loop structures, and so on.

As described herein, a “genetic component” or “genetic element” may be any coding or non-coding nucleic acid sequence. In some embodiments, a genetic component is a nucleic acid that codes for an amino acid, a peptide or a protein. Genetic components may be operons, genes, gene fragments, promoters, exons, introns, regulatory sequences, or any combination thereof. Genetic components can be as short as one or a few codons or may be longer including functional components (e.g., encoding proteins) and/or regulatory components. In some embodiments, a genetic component includes an entire open reading frame of a protein, or the entire open reading frame and one or more (or all) regulatory sequences associated therewith. One skilled in the art would appreciate that the genetic components can be viewed as modular genetic components or genetic element. For example, a genetic module can comprise a regulatory sequence or a promoter or a coding sequence or any combination thereof. In some embodiments, the genetic component includes at least two different genetic elements and at least two recombination sites. In eukaryotes, the genetic component can comprise at least three modules. For example, a genetic module can be a regulator sequence or a promoter, a coding sequence, and a polyadenlylation tail or any combination thereof In addition to the promoter and the coding sequences, the nucleic acid sequence may comprise control modules including, but not limited to a leader, a signal sequence and a transcription terminator. The leader sequence is a non-translated region operably linked to the 5′ terminus of the coding nucleic acid sequence. The signal peptide sequence codes for an amino acid sequence linked to the amino terminus of the polypeptide which directs the polypeptide into the cell's secretion pathway.

As used herein, a “heterologous nucleic acid sequence” is any sequence placed at a location where it does not normally occur. A heterologous nucleic acid sequence may comprise a sequence that does not naturally occur in a cell, or it may comprise only sequences naturally found in the cell, but placed at a non-normally occurring location in the cell. In some embodiments, the heterologous nucleic acid sequence is not an endogenous sequence. In certain embodiments, the heterologous nucleic acid sequence is an endogenous sequence that is derived from a different cell. In other embodiments, the heterologous nucleic acid sequence is a sequence that occurs naturally in a cell but is then relocated to another site where it does not naturally occur, rendering it a heterologous sequence at that new site.

As used herein, a “long non-coding RNA” or “lncRNA” refers to a transcribed RNA molecule containing greater than 200 nucleotides that do not code for protein. LncRNAs are usually located within intergenic spaces of the genome. Generally, lncRNAs are a diverse class of molecules that play a variety of roles in modulation of gene function. For example lncRNAs are known to regulate gene transcription (for example, as described by Goodrich et al. Nature Reviews Molecular Cell Biology, 7 (8): 612-6, 2006), translation (for example, as described by Tiedge et al. PNAS 88:(6): 2093-7, 1991), and epigenetic regulation (for example, as described by Wutz et al. Nature Genetics, 30 (2): 167-74, 2002). Examples of lncRNAs include, but are not limited to Kcnq1ot1, Xlsirt, Xist, ANRIL and MALAT1. Further examples of lncRNAs are described, for example, in Amaral et al. Nucleic Acids Research 39((Database issue)): D146-D151, (2010).

As used herein, “operably linked” means that expression control sequences are positioned relative to a nucleic acid of interest to initiate, regulate or otherwise control transcription of the nucleic acid of interest.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

Nucleic acid sequences that are “complementary” are those that are capable of base-pairing according to the standard Watson-Crick complementarity rules. As used herein, the term “complementary sequences” may mean nucleic acid sequences that are 100% complementarity or less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity), or may be defined as being capable of hybridizing to the comparator polynucleotides.

As used herein, the term “primer” refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of the primer. A pair of bi-directional primers consists of one forward and one reverse primer as commonly used in the art of DNA amplification such as in PCR amplification.

As used herein, the terms “promoter” or “promoter sequence” refer to a DNA sequence which when ligated to a nucleotide sequence of interest is capable of controlling the transcription of the nucleotide sequence of interest into mRNA or non-coding RNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or abiotic conditions. The promoter may be native or non-native to the cell in which it is found.

Promoters which cause a gene to be expressed in most cell types at most times are commonly referred to as “constitutive promoters”. The term “inducible promoter” refers to promoters that selectively express a coding sequence or functional RNA in response to the presence of an endogenous or exogenous stimulus, for examples, by chemical compounds (chemical inducers) or in response to environmental, hormonal, chemical, and/or developmental signals.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the cell of an organism (or the encoded protein product of that sequence) is deemed “recombinant” herein if a heterologous sequence is placed adjacent to the endogenous nucleic acid sequence, such that the expression of the endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the cell of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a cell. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the term “reporter” refers to a gene, operon, or protein that can be attached to a regulatory sequence of another gene or protein of interest, so that upon expression in a host cell or organism, the reporter can confer certain characteristics that can be relatively easily identified and/or measured. Reporter genes are often used as an indication of whether a certain gene has been introduced into or expressed in the host cell or organism. Examples of commonly used reporters include: antibiotic resistance genes, fluorescent proteins, auxotropic selection modules, β-galactosidase (encoded by the bacterial gene lacZ), luciferase (from lightning bugs), chloramphenicol acetyltransferase (CAT; from bacteria), GUS (β-glucuronidase; commonly used in plants) and green fluorescent protein (GFP; from jelly fish). Reporters or selection moduless can be selectable or screenable.

As used herein, “selection marker” refers to a gene that confers a trait suitable for artificial selection. Typically host cells expressing the selectable selection marker is protected from a selective agent that is toxic or inhibitory to cell growth. Examples of commonly used selective markers include antibiotic resistance genes and auxotropic selection modules.

As used herein, the term “transcription” refers to the synthesis of coding or non-coding RNA from a DNA template.

As used herein, the term “translation” refers to the synthesis of a polypeptide from an mRNA template.

As used herein, a “vector” refers to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

### Regulatable Yeast Expression Systems

Yeast is an excellent model system for the study of eukaryotic cell biology. Expression of proteins in yeast is also a common alternative to prokaryotic and higher eukaryotic expression. Yeast cells offer many of the advantages of producing proteins in microbes (growth speed, easy genetic manipulation, low cost media) while offering some of the attributes of higher eukaryotic systems (post translational modifications, secretory expression). Several yeast protein expression systems exist in organisms from the genera Schizosaccharomyces, Saccharomyces, Pichia, Kluyveromyces, Hansenula and Yarrowia.

Regulatable gene expression systems are major tools in understanding the function of individual genes and their products. Rapid induction kinetics, reproducible and titratable induction levels, and low reagent costs are advantageous features of expression systems for use in basic research and for industrial-scale protein production.

Due to their critical role in expression cassette design, promoters are the most characterized and engineered genetic elements in many yeast systems. Commonly used constitutive promoters in yeasts include, but are not limited to ADH1, GAPDH, PGK1, TPI, ENO, PYK1, TEF, PGK, RPS7, XPR2/hp4d, GAP and YPT1. Commonly used inducible promoters in yeasts include, but are not limited to GAL1-10, CUP1, ADH2, LAC4, ADH4, PDX2, POT1, ICL1, AOX1, FLD1 and PEX8.

In the Schizosaccharomyces pombe or “fission yeast” model, the thiamine-repressible nmt1 gene promoter is used for regulatable gene expression system. The nmt1 gene promoter is considered as an inducible/repressible strong promoter that directs the transcription. Transcription can be repressed by the addition of thiamine to a medium or induced in the absence of thiamine. Upon addition of thiamine, the nmt1 transcript level declines after 1 hour and is undetectable by 3 hours. nmt1 gene promoter has excellent dynamic range and a low off-state transcription and takes 14-16 h to induce upon thiamine withdrawal. The nmt1 gene promoter system has been adapted to overexpress genes placed under its control. However, the nmt1 gene promoter system has the following limitations: (i) it takes 14-20 hours to induce gene expression; (ii) it requires harvesting cells grown in medium with thiamine, conducting extensive washes and re-suspending in thiamine-free medium, making it cumbersome for large cultures; and (iii) it has high basal expression in the repressed state when placed on high-copy plasmid.

Other regulatable fission yeast expression systems have been developed, variously entailing glucose depletion, ethanol induction, heat-shock induction, copper-based regulation, tetracycline-induction, uracil-based regulation, estradiol-regulated induction, and pheromone induction. Examples of various regulatable promoter including but not limited to fbp1, inv1, gld1, hsp16, ctr4, tetracycline-regulatable CaMV 35S promoter, urg1 and sxa2/rep2. Many of these methods have one or more of limitations such as narrow dynamic range, strain construction, self-limiting gene expression, induction-based growth arrest, induction of stress and system-wide transcriptional changes. The present technology provides compositions and methods to address these limitations.

lncRNA Transcriptional Interference Regulates Fission Yeast Phosphate Homeostasis

Fission yeast phosphate homeostasis is achieved by transcriptional control of the PHO regulon comprising three genes—pho1, pho84 and tgp1—encoding proteins involved in extracellular phosphate acquisition. Pho1 is a cell surface acid phosphatase, Pho84 is an inorganic phosphate transporter, and Tgp1 is a glycerophosphate transporter. The expression of these genes is dependent on Pho7, a member of the Zn2Cys6 family of transcription factors. In phosphate-rich conditions, the expression of tgp1 is repressed by the transcription of a 5′ adjacent cis-acting lncRNA known as nc-tgp1 for tgp2. The model of repression is that RNA polymerase II transcribing the lncRNA traverses the promoter of the downstream tgp1 gene, thereby ejecting Pho7 from the tgp1 promoter and interfering with tgp1 expression.

lncRNA-Regulated Gene Expressions System of the Present Technology

The present disclosure provides an expression system comprising a nucleic acid sequence, wherein the nucleic acid sequence includes (a) a nc-tgp1 long noncoding RNA (lncRNA) gene that is operably linked to a thiamine responsive expression control sequence, and (b) a gene of interest that is operably linked to a tgp1 gene promoter, wherein the thiamine responsive expression control sequence is located upstream of the tgp1 gene promoter. In some embodiments, the gene of interest may be endogenous or heterologous. Additionally or alternatively, in some embodiments, the gene of interest encodes a protein, an enzyme, a structural polypeptide, a toxin, a fusion protein, an antibody agent, a drug, a cytokine, an enzyme inhibitor, a growth factor, a signaling protein, a catalytic RNA, or an inhibitory RNA (e.g., miRNA, siRNA, sgRNA, antisense oligonucleotide). In certain embodiments, the gene of interest is pho1, fkh2 or sak1.

Examples of suitable thiamine responsive expression control sequences include, but are not limited to a nmt1 promoter (Basi G, Schmid E, Maundrell K. (1993) Gene 123:131-136; Maundrell K. (1990) J Biol Chem 265:10857-10864), a Y. lipolytica P3 promoter (Walker et al., Appl Environ Microbiol. 2020 February; 86(3): e02299-19), and a Pichia pastoris THI11 promoter (Landes et al., Biotechnol Bioeng (2016) 113(12):2633-2643). In certain embodiments, the nmt1 promoter comprises the nucleic acid sequence TCCTGGCATATCATCA (SEQ ID NO: 7) or GGAAGAGGAATCCTGGCATATCATCA (SEQ ID NO: 9).

Additionally or alternatively, in some embodiments, the nc-tgp1 lncRNA gene comprises the nucleic acid sequence of SEQ ID NO: 10:

(SEQ ID NO: 10)
GGAAACTTTTTAAATTTTATTCTATTTTTCTCTTGGGCAATTTTTGCTT
TGCTGTTTGGTTTGAGGTCAGTCGCCAAAGGCAATAGCATTGTGTGCAT
GTGTGCATGGTTAAAATGAAAAGTTGGCTAACAACATTTTTATTCAAAA
ATCATTAACATACAAATCTAATCAAAAACAAAATGAATATAGTAAGCAA
CAAGAATTAAATCATTTTTTTTTCATTGTTTTAATTTTAATGAGTAATT
CCTTTCTTCAATTCCTTATTTTTTCATCCTTCCATTCATTCATTCATGC
ATTCCATCATTCCTCCTTGATGCAATGGATCGCATGGATATACATACAT
TGATTTGTGAATCGTGTGCATCTATTGATGGATGGGAGTTGTATGTGTT
TGTGTGATGAGGGTTTGTTGAAGCAAGGCATTGTCGAGGATGCCCTCTA
TTTTGTGTTGTGTTTTAGATTAAAAGTTTAATGTGTTTATCTCTTTAAT
ATACATTGTTACACAAAATCTCTGTTTATGATATTCATTTCGTTCCTAT
AGTTTATTCGAATGAATTGAATTGAATTGACTAGACTGACTATGCATAC
CAATCTTGTTGCTTCTATTGTTTAACGAAATACGCATAGAATGCTCGGA
TTGTCGACTTTATCATAGTTTTACGAAGTGAAATTTTTTTAAATGCTGC
ACTCACATACTGACTGTGTGATGTGAAAGATGAAAGTGTCATATCAAAT
TTAATCAAATGAACCCAAGGGAGAGTGAATATTATGAATTCACTCATCC
TTGATGTTGGCAGAACGAGTCGTTTTGTCTGCATATCTGAGTAACCCCC
CCGGAGCTATCCTGAGTGAAACTAAACTCGGGCATGCATTTCAACGTTT
TGCATGGTCTAATCATTTTTTGCATTGACTATATCTTTATTTGCATTTC
ACTATATTGTTTTGCACTTTACTAGATCGTTTTTGCATCTATGGTACGT
TTTGCACTAATGAATTTTTTGCACTCACTCAATTTAAACTGCTTCCAAG
AATAAAACGTTTTTATTTTTATTTTTTTTTCTTTTCCTTTGCCGTCCGT
TGTTTGTCACCCTCAACTACACAAAATATCGACTCCGTGACTGTCATGT
TTTATCTTTCACACGAATGACGATGAAATGTGACAGTTTATCTGGTCAG
GATGATTGTACAGCCTGGGCCTTGGTTTGCATACAATTGAACGAATTGT
GAGTGATTTAATGGTGGTTAATCTTAGAAATACTATAATCTACTTCAAC
CAATTCTATTACTTCATTTTTTGCTTTGCTGATACGGCATGGTTGCCTC
ATGTGTAGGACATTTGTCTGACAAACCAATTATCCCTACACGGGAAGAG
AATTCCTTGGGTTAAACGGCATGTGGTCATTATCAAGGTTTCGTTAACT
TTTGTCCCACAGGATGAGGTTGCCAAATCGTAATACCAGCCTGGGTCTC
AAGAGAGAAAATGCGACAAGGGATGCATCGGCAAATGCCAACTCAAAGA
TGTCACTTTTACAGTATTCATGAGCAATGTGGTGGTGATAGACTCAAGT
AACTGTGTTAAAATTAAGACAGGGGCCTGCTAAAATGAACTTACGAATA
AATCGCTTTTTCCTTCGGACATTCAA

Additionally or alternatively, in some embodiments, the expression levels of the protein encoded by the gene of interest may be fine-tuned by titration of thiamine concentration. Suitable thiamine concentrations may range from about 1 nM to about 50 μM. In some embodiments, the thiamine concentration ranges from about 0.01 μM to about 50 such as about 0.01 about 0.025 about 0.05 about 0.075 μM, about 0.1 μM, about 0.2 μM, about 0.5 about 0.75 about 1 about 2 about 3 about 4 about 5 about 6 about 7 about 8 about 9 about 10 about 15 about 20 about 25 about 30 about 35 about 40 about 45 or about 50 μM.

In any of the preceding embodiments of the expression system, the nc-tgp1 lncRNA gene comprises a cluster of DSR elements. In certain embodiments, the cluster of DSR elements comprises the nucleic acid sequence TTCAAACAACCCCCTTAAAACTATCTCAAACG (SEQ ID NO: 5). In other embodiments, the cluster of DSR elements is mutated and comprises the nucleic acid sequence CTGAGT (SEQ ID NO: 3) and/or CCGGAG (SEQ ID NO: 4). Additionally or alternatively, in some embodiments, the cluster of DSR elements comprises the nucleic acid sequence TCTGAGTAACCCCCCCGGAGCTATCCTGAGTG (SEQ ID NO: 6). DSR mutations have been shown to enhance lncRNA repression of the tgp1 promoter (Sanchez A. M., Shuman S., and Schwer B., RNA 24: 237-250 (2018)).

Additionally or alternatively, in some embodiments, the tgp1 gene promoter comprises the nucleic acid sequence of SEQ ID NO: 11:

(SEQ ID NO: 11)
TCGGACATTCAAATCAGTAAGTCAAAGTGTGAAAGCGTCATTGGTTGT
GTTATTCGAGACTTGTAAGTCGGCAGTAAATCTATCTGTAGCGAGTGT
GTTTGAGAAAGGAGGAAATTTTTGCATTGTTCATTAGCAGACTTGACA
TTTACCGTGTAGGTATTTAAGGTGGCAGGCTTGCCAGCTAGAAATTTC

In certain embodiments of the expression system disclosed herein, the tgp1 gene promoter comprises a Pho7 DNA binding domain. The Pho7 DNA binding domain may comprise the nucleic acid sequence of TCGGACATTCAA (SEQ ID NO: 1) or TCAGACATTCAA (SEQ ID NO: 2). It is appreciated that a mutated Pho7 DNA binding domain may lead to altered levels of induced expression of the gene of interest. Thus, by selecting different Pho7 nucleic acid sequences, the levels of induced expression of the gene of interest can be controlled.

The expression system of the present technology can be introduced as a linear construct, or a circular vector, which can be incorporated and inherited as a transgene integrated into the host genome, or as an extrachromosomal expression vector (Gassmann, et al., Proc. Natl. Acad. Sci. USA (1995) 92: 1292). The expression vector may be a DNA or RNA vector. Additionally or alternatively, in some embodiments, the expression vector is a plasmid, a cosmid, a bacmid, a bacterial artificial chromosome (BAC), a yeast artificial chromosome (YAC), or a viral vector.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses) and artificial chromosomes, which serve equivalent functions. Various vectors can be used to construct the expression system of the present technology. In certain embodiments, the vector can be a viral vector, a vector of bacterial origin (e.g., an E. coli expression vector), a mammalian vector or a yeast vector. In one embodiment, the vector is a fission yeast vector. Non-limiting examples of fission yeast vectors include pDUAL, pAL19, paR3, pBG1, pDBlet, pEA500, pFL20, pIRT2, pIRT2U, ura4+, pIRT2-CAN1, pJK148, pJK210, pON163, pNPT/ADE1-3, pSP1, pSP2, URA3, pSP3, pSP4, LYS2, pUR18, pUR19, pZA57, pWH5, pART1, pCHY21, pEVP11, REP1, REP3 and REP4. It is appreciated that different vectors may exhibit similar or different levels of induced expression of the protein of interest. Methods for producing diverse populations of vectors have been described by Lerner et al., U.S. Pat. Nos. 6,291,160 and 6,680,192.

Successful introduction of expression vectors into host cells can be monitored using various known methods. Selection of expression vectors suitable for inserting nucleic acid sequences for expressing transcripts into the vector, and methods of delivering the vector to the cells of interest are within the skill in the art. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. The delivery of the expression vector containing recombinant DNA can by performed by abiologic or biologic systems including but not limited to transformation, transduction particles derived from phage or viruses, and conjugation.

In another aspect, the present disclosure provides a host cell comprising any and all embodiments of the expression system disclosed herein. The expression systems of the present technology may be used to induce/repress expression of a gene of interest in prokaryotic or eukaryotic cells. For example, a protein of interest can be expressed in cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990).

One strategy to maximize recombinant polypeptide expression in a host cell is to express the polypeptide in a host cell with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the sequence of the nucleic acid to be inserted into an expression vector so that the individual codons for each amino acid are those that are preferentially utilized in the host cell, i.e., codon optimization. Such alteration of nucleic acid sequences may be carried out by standard DNA synthesis techniques.

Additionally or alternatively, in some embodiments, the host cell is a Saccharomyces yeast, a Schizosaccharomyces yeast, a Candida yeast, a Pichia yeast, a Hansenula yeast, a Trichosporon yeast, a Brettanomyces yeast, a Pachysolen yeast, a Yamadazyma yeast, a Kluyveromyces yeast, or a Yarrowia yeast. Non-limiting examples of yeast cells include, but are not limited to, Saccharomyces cerevisiae, Schizosaccharomyces pombe, Candida shehatae, Pichia stipites, Kluyveromyces marxianus, Kluyveromyces lactis and the like. Methods of preparing and culturing (fermenting) such yeasts are well-known to those skilled in the art, and the present production method can be implemented by a person skilled in the art with reference to known techniques. Various well-known conventional methods can be used for transformation, such as transformation methods, transfection methods, conjugation methods, protoplast methods, electroporation methods, lipofection methods, lithium acetate methods and the like, which are similarly well-known to those skilled in the art.

Without wishing to be bound by theory, it is believed that the addition of thiamine to the expression systems disclosed herein will rapidly repress expression of the nc-tgp1 lncRNA that interferes with the activity of the downstream tgp1 gene promoter, thereby leading to tgp1 gene promoter-induced expression of the gene of interest. In one aspect, the present disclosure provides a method for overexpressing a heterologous polypeptide in a eukaryotic cell comprising contacting any and all embodiments of the host cell disclosed herein with an effective amount of thiamine, wherein the heterologous polypeptide is encoded by the heterologous gene of the expression system of the present technology. In another aspect, the present disclosure provides a method for decreasing expression of a heterologous polypeptide in a eukaryotic cell comprising culturing any and all embodiments of the host cell in a thiamine-free medium, wherein the heterologous polypeptide is encoded by the heterologous gene of the expression system of the present technology. Additionally or alternatively, in some embodiments, the methods of the present technology further comprise detecting activity and/or expression levels of the heterologous polypeptide.

In any and all embodiments of the methods disclosed herein, the activity and/or expression levels of the heterologous polypeptide are detected via a functional catalytic assay (e.g., acid phosphatase activity assay), a phenotypic assay, a colorimetric assay, enzyme-linked immunosorbent assays (ELISA), dot blotting, immunofluorescence, fluorescent microscopy, immunoprecipitation, immunoelectrophoresis, flow cytometry, western blotting, HPLC, or mass-spectrometry.

### Kits of the Present Technology

The present technology provides kits for use in any of the methods described herein. In one aspect, the present disclosure provides kits including any and all embodiments of the expression systems disclosed herein and instructions for using the expression systems to express gene products encoded by the gene of interest. In one aspect, the kits may include non-endogenous expression vectors comprising a nucleic acid sequence, wherein the nucleic acid sequence includes (a) a nc-tgp1 long noncoding RNA (lncRNA) gene that is operably linked to a thiamine responsive expression control sequence, and (b) a gene of interest that is operably linked to a tgp1 gene promoter, wherein the thiamine responsive expression control sequence is located upstream of the tgp1 gene promoter. Additionally or alternatively, in some embodiments, the kits further comprise host cells, and instructions for transforming the non-endogenous expression vectors into the host cells and using the transformed cells to express gene products encoded by the gene of interest. In some embodiments, the host cells are eukaryotic cells, yeast cells, insect cells or mammalian cells. In any of the embodiments disclosed herein, the kits can also comprise, e.g., a buffering agent, a preservative, a stabilizing agent, cell culture medium, cell culture supplements and the like. The kits of the present technology can further comprise components necessary for detecting expression levels and/or activity of the gene products encoded by the gene of interest. The kits can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way.

### Example 1

### Materials and Methods

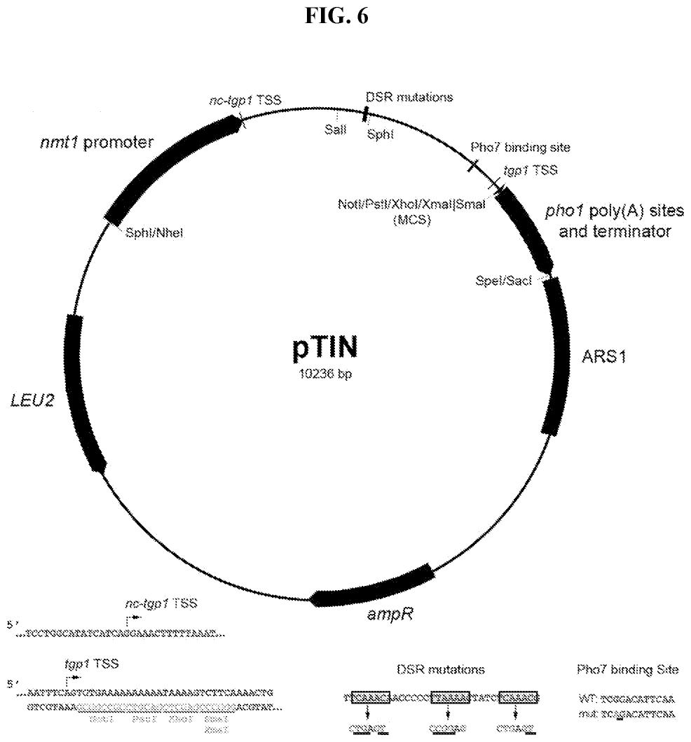

Generation of pTIN system vectors. The thiamine-inducible vector, pTIN, consists of four elements in series from 5′ to 3′ (FIG. 6): (i) the nmt1 promoter, comprising an 1108-bp segment of genomic DNA consisting of nucleotides -1177 to -70 relative to the +1 nmt⁺ translation start site; (ii) nc-tgp1 and the tgp1 promoter, an 1865-bp genomic DNA segment from the transcription start site of nc-tgp1 lncRNA to the −1 nucleotide relative to the tgp1 translation start site; mutations of a cluster of DSR (determinant of selective removal) elements were included as shown in FIG. 6; (iii) a poly-linker consisting of the restriction sites 5′-NotI, PstI, XhoI and SmaI/XmaI; and (iv) a 647-bp segment of genomic DNA 3′ of the pho1⁺ stop codon consisting of poly(A) site(s) and a transcription terminator followed by an Spel restriction site. The SacI-SphI LEU2-containing DNA fragment of pREP3X served as the plasmid backbone (Forsburg SL., Nucleic Acids Res 21: 2955-2956 (1993)). Two-stage overlap-extension PCR was used to fuse the nmt1⁺ promoter to the nc-tgp1 transcription start site. To generate pTIN7m, the alternative expression vector with lower target gene expression, the Pho7 binding site of the pTIN vector was mutated from 5′-TCGGACATTCAA (SEQ ID NO: 1) to 5′-TCAGACATTCAA (SEQ ID NO: 2). A segment of genomic DNA starting at the +1 position of the pho1 open reading frame (ORF) to 647-bp 3′ of the pho1⁺ stop codon that had the NotI and SpeI sites upstream and downstream of this region, respectively, was cloned between the aforementioned sites in the pTIN or pTIN7m vectors. The fkh2 or sak1 ORFs were PCR amplified from S. pombe cDNA so as to introduce NotI and XhoI sites immediately upstream and downstream of the translation start and stop codon, respectively, and subsequently cloned in the pTIN vector using the aforementioned sites. The promoter-less pTIN-pho1 was generated by digesting the pTIN-pho1 vector with Nhel (restriction site 5′ of the nmt1 promoter; FIG. 6) and NotI, followed by fill-in of overhangs with DNA Polymerase I Klenow fragment (NEB) and blunt-end ligation of the vector backbone. All plasmid constructs were sequenced to exclude unwanted mutations.

The DNA sequence of the pTIN plasmid is provided below:

>MT740318.1 Cloning vector pTIN, complete sequence
(SEQ ID NO: 8)
AGCGCCCAATACGCAAACCGCCTCTCCCCGCGCGTTGGCCGATTCATTAATGCAGCTGGCACGACAGGTT
TCCCGACTGGAAAGCGGGCAGTGAGCGCAACGCAATTAATGTGAGTTAGCTCACTCATTAGGCACCCCAG
GCTTTACACTTTATGCTTCCGGCTCGTATGTTGTGTGGAATTGTGAGCGGATAACAATTTCACACAGGAA
ACAGCTATGACCATGATTACGCCAAGCTTGTCGATCGACTACGTCGTTAAGGCCGTTTCTGACAGAGTAA
AATTCTTGAGGGAACTTTCACCATTATGGGAAATGGTTCAAGAAGGTATTGACTTAAACTCCATCAAATG
GTCAGGTCATTGAGTGTTTTTTATTTGTTGTATTTTTTTTTTTTTAGAGAAAATCCTCCAATATATAAAT
TAGGAATCATAGTTTCATGATTTTCTGTTACACCTAACTTTTTGTGTGGTGCCCTCCTCCTTGTCAATAT
TAATGTTAAAGTGCAATTCTTTTTCCTTATCACGTTGAGCCATTAGTATCAATTTGCTTACCTGTATTCC
TTTACATCCTCCTTTTTCTCCTTCTTGATAAATGTATGTAGATTGCGTATATAGTTTCGTCTACCCTATG
AACATATTCCATTTTGTAATTTCGTGTCGTTTCTATTATGAATTTCATTTATAAAGTTTATGTACAAATA
TCATAAAAAAAGAGAATCTTTTTAAGCAAGGATTTTCTTAACTTCTTCGGCGACAGCATCACCGACTTCC
GTGGTACTGTTGGAACCACCTAAATCACCAGTTCTGATACCTGCATCCAAAACCTTTTTAACTGCATCTT
CAATGGCCTTACCTTCTTCAGGCAAGTTCAATGACAATTTCAACATCATTGCAGCAGACAAGATAGTGGC
GATAGGGTTGACCTTATTCTTTGGCAAATCTGGAGCAGAACCGTGGCATGGTTCGTACAAACCAAATGCG
GTGTTCTTGTCTGGCAAAGAGGCCAAGGACGCAGATGGCAACAAACCCAAGGAACCTGGGATAACGGAGG
AACTAGGATCATGGCGGCAGAATCAATCAATTGATGTTGAACCTTCAATGTAGGGAATTCGTTCTTGATG
GTTTCCTCCACAGTTTTTCTCCATAATCTTGAAGAGGCCAAAACATTAGCTTTATCCAAGGACCAAATAG
GCAATGGTGGCTCATGTTGTAGGGCCATGAAAGCGGCCATTCTTGTGATTCTTTGCACTTCTGGAACGGT
GTATTGTTCACTATCCCAAGCGACACCATCACCATCGTCTTCCTTTCTCTTACCAAAGTAAATACCTCCC
ACTAATTCTCTGACAACAACGAAGTCAGTACCTTTAGCAAATTGTGGCTTGATTGGAGATAAGTCTAAAA
GAGAGTCGGATGCAAAGTTACATGGTCTTAAGTTGGCGTACAATTGAAGTTCTTTACGGATTTTTAGTAA
ACCTTGTTCAGGTCTAACACTACCGGTACCCCATTTAGGACCACCCACAGCACCTAACAAAACGGCATCA
ACCTTCTTGGAGGCTTCCAGCGCCTCATCTGGAAGTGGGACACCTGTAGCATCGATAGCAGCACCACCAA
TTAAATGATTTTCGAAATCGAACTTGACATTGGAACGAACATCAGAAATAGCTTTAAGAACCTTAATGGC
TTCGGCTGTGATTTCTTGACCAACGTGGTCACCTGGCAAAACGACGATCTTCTTAGGGGCAGACATTAGA
ATGGTATATCCTTGAAATATATATATATATATTGCTGAAATGTAAAAGGTAAGAAAAGTTAGAAAGTAAG
ACGATTGCTAACCACCTATTGGAAAAAACAATAGGTCCTTAAATAATATTGTCAACTTCAAGTATTGTGA
TGCAAGCATTTAGTCATGAACGCTTCTCTATTCTATATGAAAAGCCGGTTCCGCGGCTCTCACCTTTCCT
TTTTCTCCCAATTTTTCAGTTGAAAAAGGTATATGCGTCAGGCGACCTCTGAAATTAACAAAAAATTTCC
AGTCATCGAATTTGATTCTGTGCGATAGCGCCCCTGTGTGTTCTCGTTATGTTGAGGAAAAAAATAATGG
TTGCTAAGAGATTCGAACTCTTGCATCTTACGATACCTGAGTATTCCCACAGTTAACTGCGGTCAAGATA
TTTCTTGAATCAGGCGCCTTAGACCGCTCGGCCAAACAACCAATTACTTGTTGAGAAATAGAGTATAATT
ATCCTATAAATATAACGTTTTTGAACACACATGAACAAGGAAGTACAGGACAATTGATTTTGAAGAGAAT
GTGGATTTTGATGTAATTGTTGGGATTCCATTTTTAATAAGGCAATAATATTAGGTATGTAGATATACTA
GAAGTTCTCCTCGACAAGCTTGCATGCGCTAGCGTCGATCGACTCTAGAGGATCAGAAAATTATCGCCAT
AAAAGACAGAATAAGTCATCAGCGGTTGTTTCATTTCCTATATTTTTTTTTTATTTTTTTATTTTTTAAT
AAGGGAAAATTTAACGTCTAAGGATACAGAAGATTGTTAGCACATTAAAGTAATAAAGGCTTAAGTAGTA
AGTGCCTTAGCATGTTATTGTATTTCAAAGGACATAATCTAAAATAATAACAATATCATTTCTCACAAGT
TATTCAATTTTCTTTTTTTTTTCTAATAATATCAAGAATGTATTATTTGTTTGACATAAGTCAACTAATT
TATTTAATATGCTGGATTAATCTTGCAGACATGTAAATTAACAAGTTTTAGTCAAATAACGTTGAAGTTT
CAATGAACTCAAATAATTTCTCTTTTTTTTTATATAACCATATGTCTAATCTGATTTATATTTTCCGCAG
GGATCAACTGAAGTTATGACATTTGGATTGGATCACTTATAACCTTGGTCGCCAAATAATACAAAAATCA
GCGTTATAAAACAAAGAAGGTTTTTGTTAAGAAATTAATCCTCTTTCTTGATAAGAAAGTTGAACCGAAA
TTGCAGATACTGATATATGAAAATAATACCCACAATTTTGGGAATAGCGCAAGCCTCAATTTAAACAATA
GGTGAGGACACATGATAATGACCTCAATGATTGTTAGAAGAAAAGAGCCTCATTACAAAATCGAAAAATG
AATGGTTGGGTACAAGTTTCCAAAACATGGTAAAGTGGACTTTGCGTATGAGACGTAAATAGAAAAAAAC
ACTTGTTATATGTTTTCTAGAATTATTGTTGTCTCTTTATGGTTGGATGATGCAAAATAGTAATTTCGGT
TAGTTGCTGTAAAACACCACGAGACAAATAGATATGGATATTTATTAAATCAGGAAAAACGTAACTCTCG
GCTACTGGATGGTTCAGTCACCCAACGATTACTGGGGAGAGAAAACAGGGCAAAAGCAAAGCTTAAAGGA
ATCCGATTGTCATTCGGCAATGTGCAGCGAAACTAAAAACCGGATAATGGACCTGTTAATCGAAACATTG

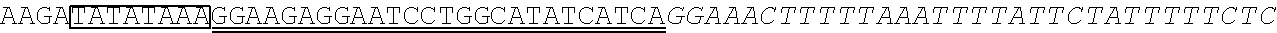

TTGGGCAATTTTTGCTTTGCTGTTTGGTTTGAGGTCAGTCGCCAAAGGCAATAGCATTGTGTGCATGTGT
GCATGGTTAAAATGAAAAGTTGGCTAACAACATTTTTATTCAAAAATCATTAACATACAAATCTAATCAA
AAACAAAATGAATATAGTAAGCAACAAGAATTAAATCATTTTTTTTTCATTGTTTTAATTTTAATGAGTA
ATTCCTTTCTTCAATTCCTTATTTTTTCATCCTTCCATTCATTCATTCATGCATTCCATCATTCCTCCTT
GATGCAATGGATCGCATGGATATACATACATTGATTTGTGAATCGTGTGCATCTATTGATGGATGGGAGT
TGTATGTGTTTGTGTGATGAGGGTTTGTTGAAGCAAGGCATTGTCGAGGATGCCCTCTATTTTGTGTTGT
GTTTTAGATTAAAAGTTTAATGTGTTTATCTCTTTAATATACATTGTTACACAAAATCTCTGTTTATGAT
ATTCATTTCGTTCCTATAGTTTATTCGAATGAATTGAATTGAATTGACTAGACTGACTATGCATACCAAT
CTTGTTGCTTCTATTGTTTAACGAAATACGCATAGAATGCTCGGATTGTCGACTTTATCATAGTTTTACG
AAGTGAAATTTTTTTAAATGCTGCACTCACATACTGACTGTGTGATGTGAAAGATGAAAGTGTCATATCA
AATTTAATCAAATGAACCCAAGGGAGAGTGAATATTATGAATTCACTCATCCTTGATGTTGGCAGAACGA
GTCGTTTTGTCTGCATATCTGAGTAACCCCCCCGGAGCTATCCTGAGTGAAACTAAACTCGGGCATGCAT
TTCAACGTTTTGCATGGTCTAATCATTTTTTGCATTGACTATATCTTTATTTGCATTTCACTATATTGTT
TTGCACTTTACTAGATCGTTTTTGCATCTATGGTACGTTTTGCACTAATGAATTTTTTGCACTCACTCAA
TTTAAACTGCTTCCAAGAATAAAACGTTTTTATTTTTATTTTTTTTTCTTTTCCTTTGCCGTCCGTTGTT
TGTCACCCTCAACTACACAAAATATCGACTCCGTGACTGTCATGTTTTATCTTTCACACGAATGACGATG
AAATGTGACAGTTTATCTGGTCAGGATGATTGTACAGCCTGGGCCTTGGTTTGCATACAATTGAACGAAT
TGTGAGTGATTTAATGGTGGTTAATCTTAGAAATACTATAATCTACTTCAACCAATTCTATTACTTCATT
TTTTGCTTTGCTGATACGGCATGGTTGCCTCATGTGTAGGACATTTGTCTGACAAACCAATTATCCCTAC
ACGGGAAGAGAATTCCTTGGGTTAAACGGCATGTGGTCATTATCAAGGTTTCGTTAACTTTTGTCCCACA
GGATGAGGTTGCCAAATCGTAATACCAGCCTGGGTCTCAAGAGAGAAAATGCGACAAGGGATGCATCGGC
AAATGCCAACTCAAAGATGTCACTTTTACAGTATTCATGAGCAATGTGGTGGTGATAGACTCAAGTAACT

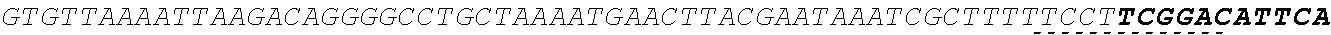

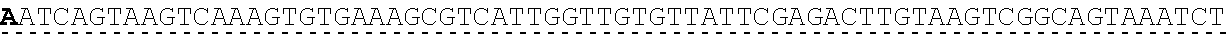

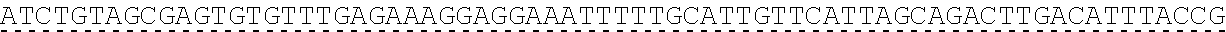

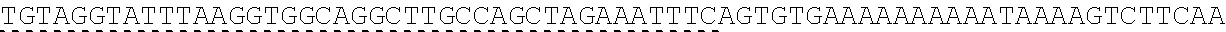

AACTGGTCGTAAAGCGGCCGCCTGCAGCTCGAGCCCGGGACGTATATCTCTTTTTAAACTTAAACCTGAA
GGTGGCTTTATCCTTGAGTCTATAATTGTTTTAGATTCCACTCTTCTATCATTTACTTTGCAACGTTCTT
TATTTTTAATAGATTGATTTTTTAAACCCTATAATTCCGGTTGTTTATAAAATGTAAGAGCTATTTGACT
TAAAGCGAAAAATTGCATAATATAAGCAGAGGATAGTTTATGTAGGAGATAATGTAATCTGCTCTATAAA
ACTGTCAAATTGTCTGTCAGAAGCACAACATTAATAAGCAGTCCGCTTTCTTACTTTAAATTCGATTAAA
CAAATATATGCGACGAGTAGAAATAAAAGCAAACTTCAACCTTTGGTTCAACATATGCTTTCTGCCGTTG
CGTGTACAAACCTATACGTGCACAACGTTGTTTAAGGTGTTTCTTTGACAATACTCTCACCATATAAAGA
TTACGATTTTTTTAGAAGAGATGACTAATTACGCGATACCTAAAAAATTTCACTCTAAGCGAATCTAAGA
TGTCCTAAGTTTCGCATATGTATTTTGGCGAAAATTGATGCCAAATCAATATTAAACAAAGAAAAACAGT
GAATAGTACAGCAAACTTGAAAAATGAAAATGGTACATTGCGCCATTTTTCGAGTTACTAGTGAGCTCGA
ATTCGAGTCTAACTCCTTAACCACTCGGACATAGTGACTTATCTGACACTCATTGTAAATTAATATATAT
ATAGGCATTTTGTTTAGTTAAAGGTACTTAAGTAATTAGTATAAACGAACCAATTTTATAATCAGGAAGT
TAAGTGAATGGTAGCACATGTCGTAAAAATTGTGAATTTTTATTGAATAATATTTTAAATACAAGCCTTT
CTAGACTAGGTATACTCATAAACATATATGAGCAAAAGGATAGAGGAGATTACATTGCATCTTCTACAAA
TTTATTTATTGCCCTTTACTGAAAAATTAAATAATGAGTACTAAATGATAAAAAGCGCTCAGTACAAGAA
AGATTGCAAAAATATTGCATTCTTCATGAATTAAAGTTGCATATAAGGCATATTGAAAGTAATAGTACTA
AAACAGCAGTTAGCGAAAATTAATAGAATTATATTCGCAAGACAATTGTGACATTAAATTAAAAAATTGT
AGAATTTTTACTATCCTCTTTAACGCCATGAGCCTTTATAAAAAGGTTAAATTAGTTTTAACATTCTTTT
TTTGAGTAAGAGTTTACAATTTATCAAAACCTGTGTTATTATATTCATTAGTTTCAATTTATTAGCATCT
AGAGAAAAATCAATTGGCAGTTACATTGTTGGAATTTATGAAGAAAAAGAATCTACAACGGAGAATAAGT
TGCTGATCGCTTTCCCTAAAATTGTATATTTTGCTGAGCTTATTTTGACATTTCGTTGAAGTTTTCTCTA
ATTCGCATTCATTTTAAGTAAAACAATGAGAAATAAAATTACAAAAAATACAAATTAAAATACAATTTTT
AGCTATAATTATAGACGATGCCCTTGTATCCCATTCTGTCTCGCTTGCCCCTACTTTTTATCTTTTATAT
ACCATAATGAACGCTGCCGCTACTAACCATACCCCGATTTTACATTTCGGACTCCCAAGGACGTACAAAA
TAGAAAACTATAGAAAAAAATAATCAGAAAATAGCATGTCATCTCTTTGTAAAACGCGTTTGCAAGAAGA
AAGGAAACAATGGAGAAGAGATCATCCATTTGTATGTAAATTTTAGTAAACTTGAAGAAATCACTAACAA
CTTCTCTTACTTAGGGATTCTATGCAAAACCTTGTAAATCATCTGATGGAGGACTCGATTTAATGAATTG
GAAGGTTGGAATTCACTGGCCGTCGTTTTACAACGTCGTGACTGGGAAAACCCTGGCGTTACCCAACTTA
ATCGCCTTGCAGCACATCCCCCTTTCGCCAGCTGGCGTAATAGCGAAGAGGCCCGCACCGATCGCCCTTC
CCAACAGTTGCGCAGCCTGAATGGCGAATGGCGCCTGATGCGGTATTTTCTCCTTACGCATCTGTGCGGT
ATTTCACACCGCATACGTCAAAGCAACCATAGTACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTTGGGTGATGGTTCACGTAGTGGGCCATCGCC
CTGATAGACGGTTTTTCGCCCTTTGACGTTGGAGTCCACGTTCTTTAATAGTGGACTCTTGTTCCAAACT
GGAACAACACTCAACCCTATCTCGGGCTATTCTTTTGATTTATAAGGGATTTTGCCGATTTCGGCCTATT
GGTTAAAAAATGAGCTGATTTAACAAAAATTTAACGCGAATTTTAACAAAATATTAACGTTTACAATTTT
ATGGTGCACTCTCAGTACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGACGAAAGGGCCTCGTGATACGCCT
ATTTTTATAGGTTAATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATGAGACAATAACCCT
GATAAATGCTTCAATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGCGGTATTATCCCGTAT
TGACGCCGGGCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAACCATGAGTG
ATAACACTGCGGCCAACTTACTTCTGACAACGATCGGAGGACCGAAGGAGCTAACCGCTTTTTTGCACAA
CATGGGGGATCATGTAACTCGCCTTGATCGTTGGGAACCGGAGCTGAATGAAGCCATACCAAACGACGAG
CGTGACACCACGATGCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCACTTCTGCGCTCGGC
CCTTCCGGCTGGCTGGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAACTGTCAGACCAAGT
TTACTCATATATACTTTAGATTGATTTAAAACTTCATTTTTAATTTAAAAGGATCTAGGTGAAGATCCTT
TTTGATAATCTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAACTCTGTAG
CACCGCCTACATACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCGGCCTTTTTAC
GGTTCCTGGCCTTTTGCTGGCCTTTTGCTCACATGTTCTTTCCTGCGTTATCCCCTGATTCTGTGGATAA
CCGTATTACCGCCTTTGAGTGAGCTGATACCGCTCGCCGCAGCCGAACGACCGAGCGCAGCGAGTCAGTG
AGCGAGGAAGCGGAAG

Double underlined—nmt1 promoter; Boxed—TATA box; Boldface—Pho7 binding site; Italics—nc-tgp1; underlined—DSR mutations;

Generation of the pREP series pho1 reporter constructs. The pho1 ORF was PCR amplified from S. pombe genomic DNA so as to introduce XhoI and XmaI sites immediately upstream and downstream from the translation start and stop codon, respectively, and subsequently cloned in pREP3X, pREP41X, or pREP81X vectors (Forsburg, Nucleic Acids Res 21: 2955-2956. 1993) using the aforementioned sites. All plasmid constructs were sequenced to exclude unwanted mutations.

Deletion of pho4 in [prt2-pho84-prt-phol]A cells. pho4 DNA segments [−452 to −1 and +1845 to +2315 relative to the pho4 translational start codon (+1)], were PCR amplified and cloned upstream and downstream, respectively, of the natMX antibiotic resistance cassette in a bacterial plasmid. The linear pho4::natMX gene disruption cassette was excised from the plasmid and transfected into haploid S. pombe [prt2-pho84-prt-pho1]Δ cells, as described by Garg A, et al., J Biol Chem 293: 4456-4467 (2018), which is hereby incorporated by reference. Nourseothricin-resistant transformants were selected and analyzed by Southern blotting to confirm correct integration at the pho4 locus.

Plasmid transformation. Plasmids were transfected by the lithium acetate method (Forsburg S L. and Rhind N., Yeast 23: 173-183 (2006), which is hereby incorporated by reference) into WT (leu1-32 ura4-D18 ade6-M216 his3-D1) or phoo4Δ pho1Δ (pho4::natMX [prt2-pho84-prt-pho2]::hygMX leu1-32 ura4-D18 ade6[M210 or M216] his3-D1) cells cultured in thiamine-free supplemented PMG (pombe minimal glutamate) medium. The plasmids must be transfected in cells cultivated in thiamine-free medium to prevent any de-repression of the target gene post-transformation due to residual intracellular thiamine. Transformants were selected on Leu⁻ PMG agar medium at 30° C.

Acid phosphatase activity assay. Aliquots of exponentially growing cultures were harvested, washed with water, and re-suspended in water. To quantify acid phosphatase activity, reaction mixtures (200 μl) containing 100 mM sodium acetate (pH 4.2), 10 mM p-nitrophenyl phosphate (NEB), and cells (ranging from 0.01 to 0.1 A₆₀₀ units) were incubated for 5 min at 30° C. The reactions were quenched by addition of 1 ml of 1 M sodium carbonate; the cells were removed by centrifugation and the absorbance of the supernatant was measured at 410 nm. Acid phosphatase activity is expressed as the ratio of A₄₁₀ (p-nitrophenol production) to A₆₀₀ (cells). The data are averages (±SEM) of at least three assays using cells from three independent cultures.

RNA analysis. Total RNA was extracted via the hot phenol method (Köhrer K. and Domdey H., Method Enzymol: 398-405 (1991), which is hereby incorporated by reference) from 3 A₆₀₀ units of harvested yeast cells. For analysis of specific transcripts by primer extension, aliquots (5 μg) of total RNA were templated for M-MuLV reverse transcriptase-catalyzed extensions of 5′ 32P-labeled oligodeoxynucleotide primers complementary to the phot and actl mRNAs in one reaction mix. The primer sequence for act1 was, 5′-GATTTCTTCTTCCATGGTCTTGTC (SEQ ID NO: 12), and for pho1, 5′-GTTGGCACAAACGACGGCC (SEQ ID NO: 13). The primer extension reactions were performed as described by Schwer B., et al., Nucleic Acids Res 26: 2050-2057 (1998), which is hereby incorporated by reference, and the products were analyzed by electrophoresis of the reaction mixtures through a 22-cm 8% polyacrylamide gel containing 7 M urea in 80 mM Tris-borate, 1.2 mM EDTA. The ³²P-labeled primer extension products were visualized by autoradiography of the dried gel. Quantification of pho1 and act1 mRNA levels was done by scanning the dried gel with a phosphorimager and analyzing the data in ImageQuant software.

Fluorescence microscopy. Aliquots of cultured cells were fixed in 70% ethanol as described in the text. Fixed cells were harvested, washed twice with water and resuspended in water. Equal volumes of the cell suspension and mounting solution [75 μg/ml DAPI (4′,6-diamidino-2-phenylindole dihydrochloride; Sigma, St. Louis, Mo.); 4.6 mg/ml Calcofluor White (Sigma, St. Louis, Mo.) in mounting medium (Vectashield H-1000; Vector Laboratories, Burlingame, Calif.)] were mixed and incubated for 5 minutes followed by slide preparation. Cells were observed by fluorescent microscopy and 30-60 independent successive frames were imaged and subsequently counted in Image.”

### Example 2

### lncRNA-Regulated Thiamine-Inducible Expression System

tgp1 is tightly repressed in phosphate-rich conditions. Repression occurs via transcriptional interference with the tgp1 promoter by synthesis in cis of an upstream adjacent nc-tgp1 lncRNA (Ard R., et al., Nat Commun 5: 5576-5584 (2014)). The nc-tgp1-tgp1 gene cassette was used to engineer a regulatable promoter system on a plasmid.

The design involves placement of the nmt1 promoter upstream of the nc-tgp1 lncRNA transcription start site (TSS), which confers thiamine-based control of nc-tgp1 lncRNA expression (FIG. 1A). Thus, in the absence of thiamine, nc-tgp1 is at peak expression, thereby repressing the downstream tgp1 promoter. The addition of thiamine represses lncRNA expression and that in turn de-represses the downstream tgp1 promoter (FIG. 1A). To increase the repressive effect of nc-tgp1 transcription, mutations were introduced in a cluster of DSR elements in the nc-tgp1 lncRNA that are binding sites for Mmil, as described by Harigaya Y., et al., Nature 442: 45-50 (2006); Chen HM., et al., PLoS One 6: e26804 (2011); Yamashita A., et al., Open Biol 2: 120014 (2012); Kilchert C., et al., Cell Rep 13: 2504-2515 (2015); and Sanchez A M., et al., RNA 24: 237-250 (2018), which are hereby incorporated by reference (FIG. 1A, FIG. 6). The DSR mutations enhance lncRNA repression of the tgp1 promoter. The lncRNA-regulated thiamine-inducible expression system will henceforth be called the pTIN system (Thiamine Inducible) and the base vector will be referred to as pTIN.

To assay the activity of this promoter system, pho1 was used as the reporter gene driven by the tgp1 promoter (FIG. 1A). Cell surface-associated Pho1 activity is readily assayable colorimetrically via the conversion ofp-nitrophenylphosphate to p-nitrophenol. The pho1 gene was cloned downstream of the tgp1 5′ UTR in the pTIN vector with its native 3′ flanking DNA to yield the pTIN-pho1 plasmid (FIG. 1A). To test background acid phosphatase levels, a promoter-less pTIN pho1 vector was constructed that lacked the DNA segment spanning the regulatory nmt1-promoternc·tgp1·tgp1 promoter region as well as the tgp1 5′ UTR and only contained the pho1 ORF. These plasmids were introduced into a strain in which the chromosomal pho1 and pho4 genes encoding the cell-surface acid phosphatases Pho1 and Pho4 were deleted. Plasmid-bearing pho1Δ pho4Δ cells were grown to mid-log phase in Leu⁻ PMG medium without thiamine and subsequently diluted and grown in Leu⁻ PMG medium with or without 15 μM thiamine. Pho1 acid phosphatase activity was measured by incubating dilutions of cells with p-nitrophenylphosphate and measuring its conversion to p-nitrophenol for 5 min at 30° C. The amount of product formed (within the linear range of detection) was normalized to cell density and is plotted on the y-axis in FIG. 1B. In Leu⁻ PMG medium with or without thiamine, the cells containing promoter-less pTIN pho1 had very low acid phosphatase activity (FIG. 1B). Cells bearing the pTIN pho1 plasmid had 3.5-fold higher acid phosphatase activity in thiamine-free medium compared to the promoter-less pTIN pho1 control. Inclusion of thiamine elicited a 58-fold increase in acid phosphatase activity (FIG. 1B).

Cultures of phot A pho4 cells bearing either the pTIN empty vector or pTIN phol were serially diluted and spotted on Leu⁻ PMG agar medium with or without 15 μM thiamine. After 3 days of incubation at 30° C., there was no difference in growth between cells harboring either the empty pTIN or the pTIN pho1 plasmid, as gauged by colony size. Colony sizes were larger on thiamine-containing medium compared to medium lacking thiamine (FIG. 1C). The plates were subsequently processed for agarose overlay acid phosphatase assay. This assay follows the acid phosphatase-catalyzed conversion of α-naphthyl phosphate to 1-naphthol, which in the presence of Fast Blue B salt forms a colored azo-dye, presenting as red coloration around the cells. pho1Δ pho4Δ cells bearing the empty pTIN plasmid remained white in the presence or absence of thiamine indicating low background signal (FIG. 1C). pho1Δ pho4Δ cells bearing pTIN pho1 were similarly white when grown on medium lacking thiamine but were intensely red when grown on medium with thiamine (FIG. 1C).

These results demonstrate that the expression systems of the present technology are useful for producing and/or overexpressing heterologous polypeptides in eukaryotic cells.

### Example 3

### Kinetics of Thiamine-Induced Gene Expression

Pho1 acid phosphatase activity in pho1Δ pho4Δ A cells bearing the pTIN pho1 plasmid was determined as a function of time after addition of thiamine (FIG. 2A). Pho1 activity increased linearly between 1 and 7 hours and continued to increase between 7 and 11 hours at 58% of the initial rate. Extending the time course of thiamine induction to 23 hours resulted in a 53-fold increase in Pho1 level over the uninduced state (FIG. 2A). To gauge the level of phol mRNA, primer extension analysis using a pho1-specific radiolabeled primer for reverse-transcription was used, in parallel with an actin gene-specific primer as a control. The phol mRNA was induced 25-fold at 30 minutes after the addition of thiamine and attained a maximum level of 61-fold by 1 hour (FIG. 2B). The levels of pho1 mRNA remained at approximately 60-fold higher than the uninduced state at all times assayed after 1 hour (FIG. 2B; time points from 4 to 9 hours not shown). The assessment of the kinetics of gene expression from the pTIN system shows that the thiamine-dependent de-repression of the tgp1 promoter allowed rapid transcriptional up-regulation, where peak levels of the pho1 mRNA were achieved by one hour. Continuous translation of the available pho1 mRNA resulted in the linear accumulation of Pho1 at the cell surface.

These results demonstrate that the expression systems of the present technology are useful for producing and/or overexpressing heterologous polypeptides in eukaryotic cells.

### Example 4

### Comparison to the Thiamine-Repressible nmt1 Expression System

To test the differences in the kinetics and absolute levels of induction of the pTIN system with the thiamine-repressed nmt1 expression system (Forsburg, Nucleic Acids Res 21: 2955-2956. 1993), acid phosphatase activity of pho1Δ pho4Δ cells bearing the pTIN-pho1, pREP3X-pho1, pREP41X-pho1, or the pREP81X-pho1 plasmid was measured as a function of time after induction (pTIN system: addition of thiamine; pREP-series: withdrawal of thiamine). In repressed conditions, the acid phosphatase activity of cells bearing pTIN-pho1 was fourfold lower than pREP3X-pho1 bearing cells, and fourfold or 15-fold higher than that of the pREP41X-pho1 or pREP81X-pho1 bearing cells, respectively (FIG. 3B). The pTIN-pho1 bearing cells induced Pho1 activity without a detectable lag period compared to the pREP3X-pho1, pREP41X-pho1, or pREP81X-pho1 bearing cells, which induced Pho1 activity after a lag of 14, 16, or 20 h, respectively (FIG. 3B). The data were fit to the Boltzmann sigmoidal model which specified the following times for half maximal induced Pho1 activity—pTIN-phol: 5.6±0.4 h, pREP3X-pho1: 22.2±0.2 h, pREP41X-phol: 24.9±0.3 h, pREP81X-phol: 25.3 ±0.4 h. The model-derived maximum induced acid phosphatase activity of the pTlNpho1 bearing cells was fourfold lower, 2.5-fold lower or equivalent to the pREP3X-pho1, pREP41X-pho1, or pREP81X-pho1 bearing cells, respectively.

These results demonstrate that the expression systems of the present technology are useful for producing and/or overexpressing heterologous polypeptides in eukaryotic cells.

### Example 5

### Effects of Thiamine Concentration

The levels of expression from the nmt1 promoter can be titrated by varying the thiamine concentration in the growth medium, as described by Javerzat J P., et al., Nucleic Acids Res 24: 4676-4683 (1996), which is hereby incorporated by reference. To test the effects of thiamine concentration on the pTIN system, pho1Δ pho4Δ cells bearing the pTIN-pho1 plasmid were propagated in Leu⁻ PMG medium either lacking thiamine or containing different concentrations of thiamine. Pho1 activity was induced by 59-fold for cells grown in Leu⁻ PMG medium with 15 μM thiamine compared to medium lacking thiamine. Cells grown in Leu⁻ PMG medium with 0.01, 0.025, 0.05, 0.075, 0.1, 0.3 and 3μM thiamine had Pho1 activities that were 8%, 16%, 33%, 54%, 73%, 93% and 96%, respectively of the level attained with 15 thiamine (FIG. 3A). Fitting the data to a dose-response curve indicated that half-maximal induction was attained at 0.069±0.005 μM thiamine and was saturated at ≥0.3 μM thiamine. Thus, expression of the target protein can be titrated across a wide range by varying the thiamine concentration in the growth medium.

These results demonstrate that the expression systems of the present technology are useful for producing and/or overexpressing heterologous polypeptides in eukaryotic cells.

### Example 6

### Effects of Pho7 Site Mutation on The pTIN System

The tgp1 promoter has one binding site for the transcription factor Pho7 and retains 15% of its activity in the absence of Pho7. A single nucleotide change of the Pho7 site from 5′-TCGGACATTCAA (SEQ ID NO: 1) to 5′-TCAGACATTCAA (SEQ ID NO: 2) severely weakens its interaction with the Pho7 DNA binding domain in vitro and reduces in vivo tgp1 promoter activity to 20% of the wild-type (Garg A., et al., Nucleic Acids Res 46: 11262-11273 (2018)).

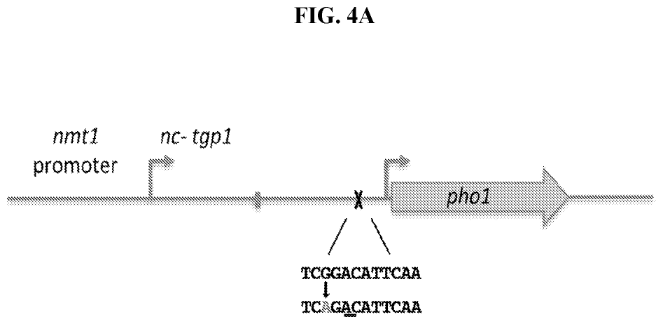

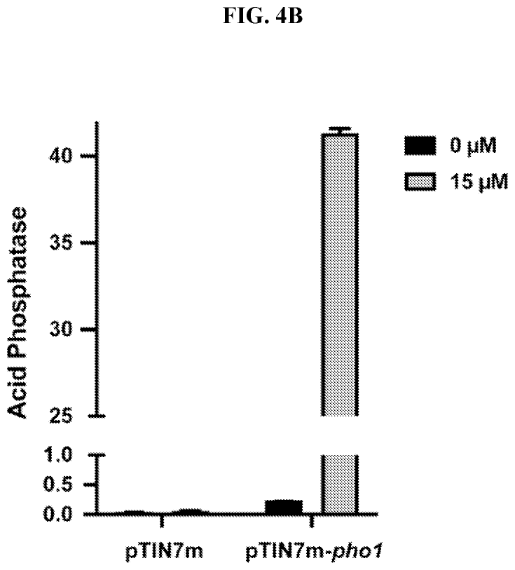

This single-nucleotide mutation was introduced at the Pho7 site in the tgp1 promoter of the pTIN plasmid, thereby generating a second-generation vector pTIN7m, into which the pho1 reporter gene was cloned (FIG. 4A). pho1Δ pho4Δ cells bearing the pTIN7m empty vector or pTIN7m pho1 plasmids were grown in Leu− PMG medium without or with thiamine and assayed for acid phosphatase activity. There was negligible Pho1 acid phosphatase generated in cells bearing the pTIN7m vector (FIG. 4B). The key findings were: (i) the uninduced level of Pho1 activity from pTIN7m pho1 was 12-fold lower than that derived from the pTIN-pho1 with an intact Pho7 site; (ii) addition of thiamine resulted in a 172-fold increase in Pho1 activity; and (iii) the level of thiamine induced Pho1 expression from pTIN7m pho1 was 24% of that derived from the pTIN pho1 (FIG. 4B, compared to FIG. 1B). These results show that the amplitude of the pTIN system can be dampened without adversely affecting the fold induction in response to thiamine.

These results demonstrate that the expression systems of the present technology are useful for producing and/or overexpressing heterologous polypeptides in eukaryotic cells.

### Example 7

### pTIN-Based Overexpression of the Mitotic Transcription Factors Sak1 and Fkh2

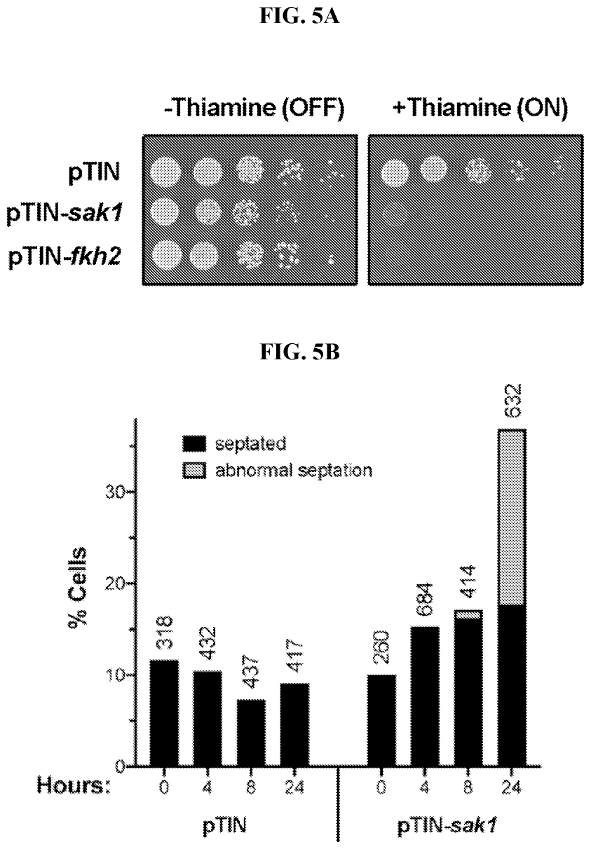

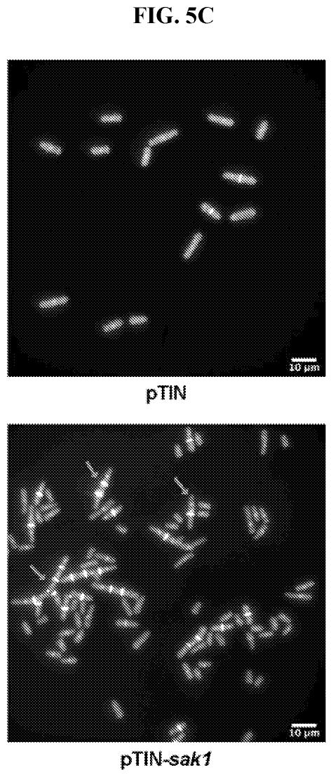

The pTIN system was exploited to overexpress the fission yeast transcription factors Sak1 and Fkh2 that regulate the mitotic (M) phase of the fission yeast cell cycle (Garg A., et al., Nucleic Acids Res 43: 6874-6888 (2015)). Overexpression of Sak1 or Fkh2 by other methods leads to abnormal cell cycle phenotypes and growth inhibition (BuckV., et al., J Cell Sci 117: 5623-5632 (2004); Vachon L., et al., Genetics 194: 873-884 (2013); Garg A., et al., Nucleic Acids Res 43: 6874-6888 (2015)). Sak1 or Fkh2 expression via the pTIN system was found to prevent growth; this was attributable to Sak1 or Fkh2 overexpression, insofar as: (i) the pTIN empty vector had no effect on growth; and (ii) growth inhibition was seen in presence of thiamine when the lncRNA-regulated tgp1 promoter was on, but not in the absence of thiamine when the lncRNA-regulated tgp1 promoter was off (FIG. 5A). Cells bearing pTIN-sak1 grew more slowly than cells with the pTIN empty vector as gauged by colony size.

Sak1 overexpressed from the nmt1 promoter system advances mitosis and leads to an increase in the septation index (percentage of cells bearing a single septum) (Garg et al. 2015). To observe phenotypes of overexpressing Sak1 from the pTIN system, cells bearing either the pTIN or pTIN-sak1 plasmid were visualized by fluorescent microscopy staining the nucleus with DAPI and the septum with Calcofluor in the uninduced state and at various times after thiamine induction (FIGS. 5B-5C). In non-inducing conditions, pTIN-sak1 bearing cells had a septation index similar to the pTIN vector control (FIG. 5B), but had an average cell length at septation that was shorter by 1.28±0.40 μm (two-tailed t-test P-value: 0.002). Thus the slower growth rate of the pTIN-sakl bearing cells in the absence of thiamine may be attributable to some degree of mitotic advancement by basal Sak1 expression.

Inducing Sak1 expression led to an increase in the septation index and a time-dependent increase in abnormal septation (percentage of septated cells that had two or more septa, mis-segregated nuclei and/or were mis-shaped) (FIGS. 5B-5C). The septation index of control cells bearing the pTIN empty vector was unaffected by thiamine (FIGS. 5B-5C). Thus, in accordance with prior observations, Sak1 overexpression-based increase in the septation index and abnormal septation indicated cell cycle defects.

These results demonstrate that the expression systems of the present technology are useful for producing and/or overexpressing heterologous polypeptides in eukaryotic cells.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An expression system comprising a nucleic acid sequence, wherein the nucleic acid sequence includes (a) a nc-tgp1 long noncoding RNA (lncRNA) gene that is operably linked to a thiamine responsive expression control sequence, and (b) a heterologous gene that is operably linked to a tgp1 gene promoter, wherein the thiamine responsive expression control sequence is located upstream of the tgp1 gene promoter.

2. The expression system of claim 1, wherein the thiamine responsive expression control sequence is a nmt1 promoter, a Y. lipolytica P3 promoter, or a Pichia pastoris THI11 promoter, optionally wherein the nmt1 promoter comprises the nucleic acid sequence TCCTGGCATATCATCA (SEQ ID NO: 7) or
(SEQ ID NO: 9)
GGAAGAGGAATCCTGGCATATCATCA.

3. (canceled)

4. The expression system of claim 1, wherein the nc-tgp1 lncRNA gene comprises the nucleic acid sequence of SEQ ID NO: 10.

5. The expression system of claim 1, wherein the nc-tgp1 lncRNA gene comprises a cluster of DSR elements.

6. The expression system of claim 5, wherein the cluster of DSR elements comprises the nucleic acid sequence
(SEQ ID NO: 5)
TTCAAACAACCCCCTTAAAACTATCTCAAACG;
or
wherein the cluster of DSR elements is mutated and comprises the nucleic acid sequence CTGAGT (SEQ ID NO: 3) and/or CCGGAG (SEQ ID NO: 4); or
wherein the cluster of DSR elements comprises the nucleic acid sequence
(SEQ ID NO: 6)
TCTGAGTAACCCCCCCGGAGCTATCCTGAGTG.

7. (canceled)

8. (canceled)

9. The expression system of claim 1, wherein the tgp1 gene promoter comprises the nucleic acid sequence of SEQ ID NO: 11.

10. The expression system of claim 1, wherein the tgp1 gene promoter comprises a Pho7 DNA binding domain, optionally wherein the Pho7 DNA binding domain comprises the nucleic acid sequence of TCGGACATTCAA (SEQ ID NO: 1) or TCAGACATTCAA (SEQ ID NO: 2).

11. (canceled)

12. The expression system of claim 1, wherein the expression system is integrated on a chromosome of a host cell.

13. The expression system of claim 1, wherein the expression system is an expression vector.

14. The expression system of claim 13, wherein the expression vector is a plasmid, a cosmid, a bacterial artificial chromosome (BAC) or a yeast artificial chromosomes (YAC).

15. The expression system of claim 1, wherein the heterologous gene encodes a protein, an enzyme, a structural polypeptide, a toxin, a fusion protein, an antibody agent, a drug, a cytokine, an enzyme inhibitor, a growth factor, a signaling protein, a bioluminescent protein, a fluorescent protein, a chemiluminescent protein, a catalytic RNA, or an inhibitory RNA.

16. The expression system of claim 15, wherein the heterologous gene is pho1, fkh2 or sak1.

17. A host cell comprising the expression system of claim 1.

18. The host cell of claim 17, wherein the host cell is a Saccharomyces yeast, a Schizosaccharomyces yeast, a Candida yeast, a Pichia yeast, a Hansenula yeast, a Trichosporonyeast, a Brettanomyces yeast, a Pachysolen yeast, a Yamadazyma yeast, a Kluyveromyces yeast, or a Yarrowia yeast.

19. The host cell of claim 17wherein the host cell is Saccharomyces cerevisiae, Schizosaccharomyces pombe, Candida shehatae, Pichia stipites, Kluyveromyces marxianus, or Kluyveromyces lactis.

20. A method for overexpressing a heterologous polypeptide in a eukaryotic cell comprising contacting a host cell comprising the expression system of claim 1 with an effective amount of thiamine, wherein the heterologous polypeptide is encoded by the heterologous gene of the expression system.

21. A method for decreasing expression of a heterologous polypeptide in a eukaryotic cell comprising culturing a host cell comprising the expression system of claim 1 in a thiamine-free medium, wherein the heterologous polypeptide is encoded by the heterologous gene of the expression system.

22. The method of claim 20, further comprising detecting activity and/or expression levels of the heterologous polypeptide.

23. The method of claim 22, wherein the activity and/or expression levels of the heterologous polypeptide are detected via a functional catalytic assay, a phenotypic assay, a colorimetric assay, enzyme-linked immunosorbent assays (ELISA), dot blotting, immunofluorescence, fluorescent microscopy, immunoprecipitation, immunoelectrophoresis, flow cytometry, western blotting, or mass-spectrometry.

24. A kit comprising the expression system of claim 1, and instructions for use.
